(19) United States
(12) Patent Application Publication
(10) Pub. No.: US 2012/0283174 A1
(43) Pub. Date: Nov. 8, 2012

(54) ANTIBIOFILM GLYCOPEPTIDES

(75) Inventors: Eric Charles Reynolds, Carlton (AU); Stuart Geoffrey Dashper, Carlton (AU)

(73) Assignee: ORAL HEALTH AUSTRALIA PTY. LTD., CARLTON, VICTORIA (AU)

(21) Appl. No.: 13/509,255

(22) PCT Filed: Nov. 11, 2010
(86) PCT No.: PCT/AU2010/001506

§ 371 Date: Jul. 18, 2012

(30) Foreign Application Priority Data
Nov. 11, 2009 (AU) 2009905526

(51) Int. Cl. A61K 38/17; A61P 31/04; A61P 1/02

(52) U.S. Cl. 514/2.4

(57) ABSTRACT

The present invention relates to peptides and compositions that have antibiofilm properties. In particular, the peptides and compositions of the invention can be used for the treatment or prevention of various conditions including dental caries, gingivitis, periodontitis, oral mucositis, dry mouth and xerostomia.

### FIELD OF THE INVENTION

The present invention relates to peptides and compositions that have antibiofilm properties. In particular, the peptides and compositions of the invention can be used for the treatment or prevention of various conditions including dental caries, gingivitis, periodontitis, oral mucositis, dry mouth and xerostomia.

### BACKGROUND OF THE INVENTION

The oral cavity is a fertile environment for the growth of bacteria with a range of hard and soft tissue surfaces that provide a variety of distinctly different microhebitats. The unique, non-shedding hard surfaces of teeth in particular, allow for accretion of the thick, complex, structured polymicrobial biofilms known as dental plaque from which more than 500 bacterial species have been identified. The stability of oral microbial biofilms requires dynamic balances by a range of synergistic and antagonistic interactions among species and the environment they create. Minor adjustments in the oral environment can affect these natural balances potentially leading to shifts in the ecology and changes in the species composition of oral microbial biofilms. These changes in species composition can lead to the development of more pathogenic supra- and subgingival plaques if opportunistic species become dominant in the microbial community. For example, increased dental caries incidence is often caused by increased consumption of dietary carbohydrates, which is linked to the acidification of fluids at the tooth surface due to the bacterial fermentation of these carbohydrates. The dietary consumption of mono- and disaccharides favors oral streptococci, such as Streptococcus mutans, that can rapidly ferment these substrates. The resultant acidic environment further promotes the proliferation of aciduric species, such as S. mutans and lactobacilli. When the plaque pH is maintained close to neutrality, especially in the absence of excess free carbohydrates, acidogenic oral bacteria such as Lactobacillus casei and S. mutans remain at low levels while proportions of more commensal species such as Actinomyces spp. and Streptococcus sanguis are relatively high.

Bacterial biofilms are ubiquitous in nature and are usually defined as matrix-enclosed bacterial populations adherent to each other and/or to surfaces or interfaces. Bacterial biofilm formation is an extremely common phenomenon with a major economic impact in different industrial, medical and environmental fields. Biofilms can comprise a single species or multiple species and can form on a wide range of abiotic and biotic surfaces and interfaces. Although polymicrobial biofilms predominate in most situations single species biofilms can occur under certain circumstances and are an increasing problem on the surface of medical implants. Growth as a biofilm offers a number of significant advantages to the bacterium over planktonic growth not the least of which is the attachment to the surface that enables the bacterium to localize itself in a favourable environment. In polymicrobial biofilms metabolic activities can be integrated and the presence of a variety of species allows for greater flexibility in metabolic and catabolic activities as the ‘genome’ of the biofilm population increases with increasing species diversity. The Centers for. Disease Control and Prevention estimate that 65% of human bacterial infections involve biofilms. Biofilms often complicate treatment of chronic infections by protecting bacteria from the immune system, decreasing antibiotic efficacy and dispersing planktonic cells to distant sites that can aid reinfection. Bacterial cells within a biofilm have been shown to be up to 500 times more resistant to certain antimicrobial agents than planktonic cells which is achieved by a number of processes including, the slowing of penetration of some antimicrobial agents into the biofilm matrix, the slowing of the growth rate of bacteria in the deeper layers of the biofilm and the binding of some antimicrobial agents to extracellular polymers thereby reducing the effective concentration. In addition, microbial biofilms have been described as microbial landscapes, which have a topography that protects against shear stress whilst allowing mass transfer. Most importantly in the oral cavity failure to attach and grow as a biofilm will rapidly result in clearance.

Many streptococci are known to form biofilms; however, the relationship between the pathogenic state and the biofilm mode of growth has been most clearly established with the oral streptococci, which are known to initiate dental caries when the bacteria are living in the biofilm environment of supragingival dental plaque. Streptococci are ubiquitous parasites of humans. Some are part of the indigenous microbiota that are involved in opportunistic infections such as dental caries and others are exogenous pathogens that cause infections ranging from mild respiratory or skin diseases to life-threatening conditions such as pneumonia, septic shock, and necrotizing fasciitis.

Chronic periodontitis is an inflammatory condition involving a host response to bacterial components that have diffused into the subjacent gingival tissue from the subgingival plaque biofilm. Specific periodontal pathogens can establish in the subgingival plaque biofilm and these species are strongly associated with disease progression. Examples of these pathogenic species include; Porphyromonas gingivalis, Tannerella forsythia, Treponema denticola and Aggregatibacter actinomycetemcomitans. The inability of the host immune system to remove the biofilm (as it is external to the tissue and accreted on a non-shedding tooth root surface) is believed to result in continual external stimulation, leading to a chronic inflammatory state. This chronic inflammation leads to periodontal tissue damage, including bone resorption caused by the cells and molecules of the host system response. The disease is . a major public health problem in all societies and is estimated to affect up to 15% of the adult population with severe forms affecting 5-6%.

The cationic antimicrobial agent Chlorhexidine is commonly used in commercial mouth rinses for anti-plaque purposes and works optimally at slightly acidic pH in the range from 5.5 to 7.0.

Non-glycosylated phosphorylated forms of bovine caseinomacropeptide (CMP) and fragments of CMP have been shown to have antibacterial activity in vitro against both Gram-negative and Gram-positive oral bacteria that are in a planktonic state (WO 1999/026971). A composition including a divalent cation and a non-glycosylated phosphorylated form of bovine CMP fragment also exhibited antibacterial activity towards bacteria that are in a planktonic state (WO 2005/058344). These peptides reduce the viability of a bacterium. There exists a need for better or alternative compositions to those currently used to reduce the pathological consequences of biofilms by preventing biofilm formation and development and also by causing the dispersion of existing biofilms.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

In one aspect, the present invention provides a peptide in an effective amount for inhibiting or reducing biofilm formation or biofilm growth, the peptide having at least one amino acid that is glycosylated and has an amino acid sequence of, or functionally similar to, a casein or fragment of a casein. Preferably, the casein is κ-casein (kappa-casein). The casein may be of bovine origin however casein derived from other animals is also included. In addition, the peptide may be derived from a genetic variant of casein or κ-casein. In preferred embodiments, the peptide has at least one amino acid that is phosphorylated.

The fragment of a casein is greater than 10, preferably greater than 20 amino acids in length and has at least 70%, 80%, 85%, 90%, 91%, 92%, 93%, 94%, 95%, 96%, 97%, 98%, 99% or 100% sequence identity to a portion of a naturally occurring casein. . Preferably, the casein is bovine casein and preferably the fragment of the casein is taken from within 106 to 169 of bovine casein (numbering as per bovine κ-casein A (SEQ ID NO: 11)). In one embodiment, the fragment is generated by trypsin or chymosin digestion of κ-casein. In other words, a peptide of the invention is κ-casein 106-169 or fragments thereof, in particular trypsin or chymosin generated fragments. In one embodiment the fragment of casein is less than 100, 90, 80, 70, 60, 50, 40 or 30 amino acids in length.

Preferably, the peptide is in a composition which further comprises a cation. Preferably, the cation is a divalent cation. The divalent cation is preferably selected from the group consisting of Zn²⁺, Ca²⁺, Cu²⁺, Ni²⁺, Co²⁺, Fe²⁺, Sn²⁺ and Mn²⁺. In addition the cation may be an ion pair SnF⁺, CuF⁺ and CaF⁺. Preferably, the divalent cation is Zn²⁺ or Ca²⁺.

Most preferably, the divalent cation is Zn²⁺. In one embodiment, the cation is water soluble in the salt form in which it is provided.

Some reports indicate that some cations have relevant activity themselves. In one embodiment, the peptide and cation are present in the composition in amounts effective to exhibit synergistic or additive bactericidal, antibiofilm or biofilm disrupting activity. In another embodiment, the composition including a peptide of the invention and a cation has a bactericidal, antibiofilm or biofilm disrupting activity that is greater than the activity exhibited by either the peptide or cation alone.

Preferably, the cation is bound to the peptide. Glycopeptides of the invention can inhibit, reduce or prevent bacterial biofilm formation or development and cause biofilm dispersion, but due to the negative charge of the glycopeptide it may be repelled by the negatively charged bacteria and the negatively charged biofilm. Without being bound by any theory, the addition of a cation, particularly zinc ions, appears to enhance the interaction of the glycopeptide with the biofilm and therefore substantially enhance activity.

In certain embodiments, the composition further comprises a peptide that is non-glycosylated, that has at least one amino acid that has been phosphorylated and has an amino acid sequence of, or functionally similar to, a casein or fragment of a casein, preferably K-casein.

In certain embodiments, the .peptides of a composition of the invention consist essentially of glycosylated peptides. These “glycosylated peptides”, defined further below, have at least one amino acid that is glycosylated. Preferably, at least 50%, 60%, 70%, 80%, 90%, 91%, 92%, 93%, 94%, 95%, 96%, 97%, 98%; 99% or 100% of the peptides in the composition are glycosylated.

In one aspect, the present invention provides a composition consisting essentially of the cation (as above) and a peptide, wherein the peptide has at least one amino acid that is glycosylated and has a sequence of, or functionally similar to, a casein or fragment of a casein, the composition having an effective amount of peptide for reducing or inhibiting biofilm formation. The casein may be of bovine origin however casein derived from other animals is also included. In addition, the peptide may be derived from a genetic variant of casein or κ-casein. In preferred embodiments, the peptide has at least one phosphorylated amino acid.

In a composition of this embodiment, the peptides in the composition are glycosylated peptides that have at least one glycosylated amino acid, such that the composition does not contain any detectable amount of non-glycosylated peptides. Detection may be by HPLC, mass spectrometry or carbohydrate stain independently (i.e. any method alone may be used).

In one embodiment, the glycosylated peptide comprises a fragment of the amino acid sequence of casein from within amino acids 106 to 169 (numbering as per bovine κ-casein A (SEQ ID NO: 11)). In another embodiment the glycosylated peptide comprises an amino acid sequence selected from the group consisting of:

(SEQ ID NO: 1)
Ala Val Glu Ser Thr Val Ala Thr Leu Glu Ala Ser(P) Pro Glu Val Ile Glu Ser Pro Pro Glu,;
(SEQ ID NO: 2)
Ala Val Glu Ser Thr Val Ala Thr Leu Glu Asp Ser(P) Pro Glu Val Ile Glu Ser Pro Pro Glu,;

and conservative substitutions therein.

In a further embodiment the glycosylated peptide comprises an amino acid sequence selected from the group consisting of:

(SEQ ID NO: 3)
Met Ala Ile Pro Pro Lys Lys Asn Gln Asp Lys Thr Glu Ile Pro Thr Ile Asn Thr Ile Ala Ser
Gly Glu Pro Thr Ser Thr Pro Thr Ile Glu Ala Val Glu Ser Thr Val Ala Thr Leu Glu Ala Ser
(P) Pro Glu Val Ile Glu Ser Pro Pro Glu Ile Asn Thr Val Gln Val Thr Ser Thr Ala Val;
(SEQ ID NO: 4)
Met Ala Ile Pro Pro Lys Lys Asn Gln Asp Lys Thr Glu Ile Pro Thr Ile Asn Thr Ile Ala Ser
(P) Gly Glu Pro Thr Ser Thr Pro Thr Ile Glu Ala Val Glu Ser Thr Val Ala Thr Leu Glu Ala
Ser (P) Pro Glu Val Ile Glu Ser Pro Pro Glu Ile Asn Thr Val Gln Val Thr Ser Thr Ala Val;
(SEQ ID NO: 5)
Met Ala Ile Pro Pro Lys Lys Asn Gln Asp Lys Thr Glu Ile Pro Thr Ile Asn Thr Ile Ala Ser
Gly Glu Pro Thr Ser Thr Pro Thr Thr Glu Ala Val Glu Ser Thr Val Ala Thr Leu Glu Asp
Ser (P) Pro Glu Val Ile Glu Ser Pro Pro Glu Ile Asn Thr Val Gln Val Thr Ser Thr Ala Val;
(SEQ ID NO: 6)
Met Ala Ile Pro Pro Lys Lys Asn Gln Asp Lys Thr Glu Ile Pro Thr Ile Asn Thr Ile Ala Ser
(P) Gly Glu Pro Thr Ser Thr Pro Thr Thr Glu Ala Val Glu Ser Thr Val Ala Thr Leu Glu
Asp Ser (P) Pro Glu Val Ile Glu Ser Pro Pro Glu Ile Asn Thr Val Gln Val Thr Ser Thr Ala
Val;
(SEQ ID NO: 7)
Thr Glu Ile Pro Thr Ile Asn Thr Ile Ala Ser Gly Glu Pro Thr Ser Thr Pro Thr Ile Glu Ala
Val Glu Ser Thr Val Ala Thr Leu Glu Ala Ser (P) Pro Glu Val Ile Glu Ser Pro Pro Glu Ile
Asn Thr Val Gln Val Thr Ser Thr Ala Val;
(SEQ ID NO: 8)
Thr Glu Ile Pro Thr Ile Asn Thr Ile Ala Ser (P) Gly Glu Pro Thr Ser Thr Pro Thr Ile Glu
Ala Val Glu Ser Thr Val Ala Thr Leu Glu Ala Ser (P) Pro Glu Val Ile Glu Ser Pro Pro Glu
Ile Asn Thr Val Gln Val Thr Ser Thr Ala Val;
(SEQ ID NO: 9)
Thr Glu Ile Pro Thr Ile Asn Thr Ile Ala Ser Gly Glu Pro Thr Ser Thr Pro Thr Thr Glu Ala
Val Glu Ser Thr Val Ala Thr Leu Glu Asp Ser (P) Pro Glu Val Ile Glu Ser Pro Pro Glu Ile
Asn Thr Val Gln Val Thr Ser Thr Ala Val;
and
(SEQ ID NO: 10)
Thr Glu Ile Pro Thr Ile Asn Thr Ile Ala Ser (P) Gly Glu Pro Thr Ser Thr Pro Thr Thr Glu
Ala Val Glu Ser Thr Val Ala Thr Leu Glu Asp Ser (P) Pro Glu Val Ile Glu Ser Pro Pro Glu
Ile Asn Thr Val Gln Val Thr Ser Thr Ala Val.

(P) designates that the preceding amino acid is phosphorylated i.e. Ser (P) is a serine amino acid that is phosphorylated. Non-phosphorylated versions of the above listed peptides are also within the scope of the invention. Subject to the requirement for peptides of the invention to have at least one amino acid that is glycosylated, within the scope of the invention are peptides with the sequences as described above and elsewhere herein with or without any other post-translational modifications.

In one embodiment, the glycosylated peptide comprises an amino acid sequence that is at least 50%, 60%, 70%, 80%, 90%, 91%, 92%, 93%, 94%, 95%, 96%, 97%, 98%, 99% identical to any one of SEQ ID NO: 1 to 10.

In a further embodiment, the glycosylated peptide consists of an amino acid sequence selected from the group consisting of:

(SEQ ID NO: 1)
Ala Val Glu Ser Thr Val Ala Thr Leu Glu Ala Ser(P) Pro Glu Val Ile Glu Ser Pro Pro Glu,;
(SEQ ID NO: 2)
Ala Val Glu Ser Thr Val Ala Thr Leu Glu Asp Ser(P) Pro Glu Val Ile Glu Ser Pro Pro Glu,;
(SEQ ID NO: 3)
Met Ala Ile Pro Pro Lys Lys Asn Gln Asp Lys Thr Glu Ile Pro Thr Ile Asn Thr Ile Ala Ser
Gly Glu Pro Thr Ser Thr Pro Thr Ile Glu Ala Val Glu Ser Thr Val Ala Thr Leu Glu Ala Ser
(P) Pro Glu Val Ile Glu Ser Pro Pro Glu Ile Asn Thr Val Gln Val Thr Ser Thr Ala Val;
(SEQ ID NO: 4)
Met Ala Ile Pro Pro Lys Lys Asn Gln Asp Lys Thr Glu Ile Pro Thr Ile Asn Thr Ile Ala Ser
(P) Gly Glu Pro Thr Ser Thr Pro Thr Ile Glu Ala Val Glu Ser Thr Val Ala Thr Leu Glu Ala
Ser (P) Pro Glu Val Ile Glu Ser Pro Pro Glu Ile Asn Thr Val Gln Val Thr Ser Thr Ala Val;
(SEQ ID NO: 5)
Met Ala Ile Pro Pro Lys Lys Asn Gln Asp Lys Thr Glu Ile Pro Thr Ile Asn Thr Ile Ala Ser
Gly Glu Pro Thr Ser Thr Pro Thr Thr Glu Ala Val Glu Ser Thr Val Ala Thr Leu Glu Asp
Ser (P) Pro Glu Val lle Glu Ser Pro Pro Glu Ile Asn Thr Val Gln Val Thr Ser Thr Ala Val;
(SEQ ID NO: 6)
Met Ala Ile Pro Pro Lys Lys Asn Gln Asp Lys Thr Glu Ile Pro Thr Ile Asn Thr Ile Ala Ser
(P) Gly Glu Pro Thr Ser Thr Pro Thr Thr Glu Ala Val Glu Ser Thr Val Ala Thr Leu Glu
Asp Ser (P) Pro Glu Val Ile Glu Ser Pro Pro Glu Ile Asn Thr Val Gln Val Thr Ser Thr Ala
Val;
(SEQ ID NO: 7)
Thr Glu Ile Pro Thr Ile Asn Thr Ile Ala Ser Gly Glu Pro Thr Ser Thr Pro Thr Ile Glu Ala
Val Glu Ser Thr Val Ala Thr Leu Glu Ala Ser (P) Pro Glu Val Ile Glu Ser Pro Pro Glu Ile
Asn Thr Val Gln Val Thr Ser Thr Ala Val;
(SEQ ID NO: 8)
Thr Glu Ile Pro Thr Ile Asn Thr Ile Ala Ser (P) Gly Glu Pro Thr Ser Thr Pro Thr Ile Glu
Ala Val Glu Ser Thr Val Ala Thr Leu Glu Ala Ser (P) Pro Glu Val Ile Glu Ser Pro Pro Glu
Ile Asn Thr Val Gln Val Thr Ser Thr Ala Val;
(SEQ ID NO: 9)
Thr Glu Ile Pro Thr Ile Asn Thr Ile Ala Ser Gly Glu Pro Thr Ser Thr Pro Thr Thr Glu Ala
Val Glu Ser Thr Val Ala Thr Leu Glu Asp Ser (P) Pro Glu Val Ile Glu Ser Pro Pro Glu Ile
Asn Thr Val Gln Val Thr Ser Thr Ala Val;
and
(SEQ ID NO: 10)
Thr Glu Ile Pro Thr Ile Asn Thr Ile Ala Ser (P) Gly Glu Pro Thr Ser Thr Pro Thr Thr Glu
Ala Val Glu Ser Thr Val Ala Thr Leu Glu Asp Ser (P) Pro Glu Val Ile Glu Ser Pro Pro Glu
Ile Asn Thr Val Gln Val Thr Ser Thr Ala Val.

(P) designates that the preceding amino acid is phosphorylated i.e. Ser (P) is a serine amino acid that .is phosphorylated. Non-phosphorylated versions of the above listed peptides are also within the scope of the invention. Subject to the requirement for peptides of the invention to have at least one amino acid that is glycosylated, within the scope of the invention are peptides with the sequences as described above and elsewhere herein with or without any other post-translational modifications.

A peptide of the invention may be isolated, purified, enriched, synthetic or recombinant. In one embodiment, the peptide is derived from milk, preferably bovine milk, or from an extract of milk.

A composition of the invention may include, consist essentially of, or consist of one or more peptides having one or more of the amino acid sequences shown in SEQ ID NO: 1 to 10 or any fragment or variants of the amino acid sequences of SEQ ID NO: 1 to 10 described herein.

In one embodiment, there is provided a therapeutic composition for treating biofilms comprising a therapeutically effective amount of peptides each having a sequence which is, or is functionally equivalent to, trypsin or chymosin digests of glycosylated casein fragments isolatable from milk.

In another embodiment, there is provided a therapeutic composition for treating biofilms consisting essentially of peptides each having at least one, amino acid that is glycosylated and each having an amino acid sequence of, or functionally similar to, a casein or fragment of a casein and a pharmaceutically acceptable carrier. Preferably at least one amino acid residue of the peptide is phosphorylated. Typically, the casein is κ-casein. In certain embodiments, the composition further comprises a divalent cation.

It is further preferred that the molar ratio of the divalent cation to the peptide is in the range of 0.5:1.0 to 15.0:1.0, preferably in the range of 0.5:1.0 to 4.0:1.0. It is further preferred that the molar ratio of the divalent cation to the peptide is in the range of 1.0:1.0 to 4.0:1.0, preferably 1.0:1.0 to 2.0:1.0.

In one embodiment, the present invention provides a composition for preventing, inhibiting or reducing biofilm formation and/or development consisting essentially of a cation and a peptide, wherein the peptide has at least one glycosylated amino acid, at least one phosphorylated amino acid and has an amino acid sequence of, or functionally similar to, a casein or fragment (as defined above) of a casein.

In one embodiment, there is provided a composition for the prevention or treatment of dental plaque, gingivitis, periodontal disease, dental caries, dry mouth or xerostomia consisting of a cation and a peptide, wherein the peptide has at least one glycosylated amino acid, at least one phosphorylated amino acid and has an amino acid sequence of, or functionally similar to, a casein or fragment (as defined above) of a casein.

In another aspect, the invention provides a use of a composition of the invention in the preparation of a medicament for the treatment or prevention of periodontal disease. Other conditions suitable for treatment or prevention are dental plaque, gingivitis, periodontitis, dental caries, oral mucositis, dry mouth and xerostomia.

In another aspect, the invention provides a use of a composition of the invention to prevent or inhibit the formation or growth of a biofilm including or consisting of a periodontal pathogen. Typically, a periodontal pathogen includes Porphyromonas gingivalis, Tannerella forsythia, Treponema denticola and Aggregatibacter actinomycetemcomitans. The biofilm may occur anywhere in the body of a subject including on any naturally occurring or implanted surface. The biofilm comprises a periodontal pathogen and may contribute to or cause many indications.

The invention also provides a use of a composition of the invention in the preparation of a medicament for the treatment or prevention of a systemic disease associated with periodontal disease. Typically, the periodontal disease or associated systemic disease may be caused by a biofilm including one or more of Porphyromonas gingivalis, Tannerella forsythia, Treponema denticola and Aggregatibacter actinomycetemcomitans.

In one embodiment; the invention provides a method of preventing or treating. a disease associated with a periodontal pathogen comprising administering to a subject in need thereof a composition of the invention. Typically, the disease would be contributed to or caused by bacteria, especially bacteria in a biofilm and disruption or inhibition of formation or growth of that biofilm treats or aids in the treatment of the disease.

In another aspect, the invention provides a use of a composition of the invention in the preparation of a medicament for the treatment or prevention of endodontic treatment failure associated with formation or growth of a biofilm on a root canal. The endodontic treatment failure may occur due to infection or reinfection with one or more bacteria leading to formation and/or growth of a biofilm. Typically, the bacteria is Enterococcus faecalis.

In one embodiment, there is provided a method of preventing or treating endodontic treatment failure comprising administering to a subject in need thereof a composition of the invention.

In another aspect, the invention provides the use of a composition of the invention as an oral lubricant, saliva substitute or as artificial saliva.

In another aspect, the invention provides an oral lubricant, saliva substitute or artificial saliva comprising a peptide or composition of the invention.

In one embodiment, the invention provides a saliva substitute, oral lubricant or artificial saliva having a therapeutically effective amount of peptides, each consisting essentially of an amino acid sequence of, or functionally similar to, a casein or fragment of a casein and each having at least one amino acid that is glycosylated.

In another embodiment the invention provides a saliva substitute, oral lubricant or artificial saliva comprising a therapeutically effective amount of an active ingredient, the active ingredient consisting essentially of peptides or a composition of the invention.

In another aspect; the invention provides a method of treating dry mouth or xerostomia comprising administering one or more peptides or compositions of the invention.

In another aspect, the invention provides a method of treating dry mouth or xerostomia comprising administering an oral lubricant, saliva substitute or artificial saliva including one or more peptides or compositions of the invention.

In another aspect, the invention provides a method of treating a symptom, or a disease or a condition associated with, dry mouth or xerostomia comprising administering one or more peptides or compositions of the invention or an oral lubricant, saliva substitute or artificial saliva including one or more peptides or compositions of the, invention.

In another embodiment the invention provides a composition for the treatment or prevention of periodontal disease (and/or the other conditions identified herein as suitable for treatment) consisting of an active ingredient of a cation and a peptide, wherein the peptide has at least one glycosylated amino acid, at least one phosphorylated amino acid and has an amino acid sequence of, or functionally similar to, a casein or fragment (as defined above) of a casein.

In another embodiment the invention provides a composition comprising a cation and a peptide, wherein the peptide has at least one glycosylated amino acid, at least one phosphorylated amino acid and has an amino acid sequence of, or functionally similar to, a casein or fragment (as defined above) of. a casein for use in the treatment or prevention of periodontal disease (and/or the other conditions identified herein as suitable for treatment).

In another embodiment the invention provides a composition consisting essentially of a cation and a peptide, wherein the peptide has at least one glycosylated amino acid, at least one phosphorylated amino acid and has an amino acid sequence of, or functionally similar to, a casein or fragment (as defined above) of a casein for use as a medicament.

The present invention also provides a pharmaceutical composition for the treatment or prevention of periodontal disease (and/or the other conditions identified herein as suitable for treatment) comprising a composition of the invention and a pharmaceutically acceptable carrier, excipient or diluent. The composition may further include an agent selected from the group consisting of anti-inflammatory agents and antibiotics. The antibiotic may be selected from the group consisting of amoxicillin, doxycycline and metronidazole.

In another embodiment the invention provides a pharmaceutical composition comprising an effective amount of peptides or a composition of the invention as a main ingredient.

In another embodiment the invention provides a pharmaceutical composition comprising a therapeutically effective amount of an active ingredient, the active ingredient consisting essentially of peptides or a composition of the invention.

In a further aspect, the invention provides a method of preventing or treating periodontal disease comprising the step of administering to a subject a composition of the invention as described in this specification. Other conditions suitable for treatment or prevention are dental plaque, gingivitis, periodontitis, dental caries, dry mouth and xerostomia. In the case of a method of the invention for preventing periodontal disease the subject may be a subject identified as being at risk of developing periodontal disease.

In one embodiment, the composition of the invention is administered directly to the gums and/or periodontal pockets of the subject. The composition may be a part of a composition applicable to the mouth such as dentifrice including toothpastes, toothpowders and liquid dentifrices, mouthwashes, troches, chewing gums, dental pastes, gingival massage creams, gargle tablets, dairy products and other foodstuffs.

The subject in need of treatment or at risk of developing periodontal disease is an animal. Preferably, the subject is a human or a dog.

In another embodiment the method of the invention further comprises the step of administering an agent selected from the group consisting of anti-inflammatory agents and antibiotics. The antibiotic may be selected from the group consisting of amoxicillin, doxycycline and metronidazole. Anti-inflammatory agents include Nonsteroidal Anti-inflammatory Drugs (NSAIDs). Examples of NSAIDs include compounds than inhibit a cyclooxygenase. Specific examples of NSAIDs include aspirin, ibuprofen and naproxen.

In another embodiment the invention provides a method of reducing biofilm thickness comprising administering to a biofilm a composition of the invention.

In another embodiment the invention provides a method of disrupting a biofilm comprising administering to a biofilm a composition of the invention.

In another embodiment the invention provides a method of increasing the susceptibility of bacteria in a biofilm to bactericidal agents comprising administering to the biofilm a composition of the invention. Preferably, the method comprises a subsequent and further step of administering a bactericidal agent.

In another embodiment the invention provides a method increasing the efficacy of a bactericidal agent comprising administering to a biofilm a composition of the invention in conjunction with or prior to administration of the bactericidal agent.

In another embodiment the invention provides a method of increasing the surface roughness of a biofilm comprising administering to the biofilm a composition of the invention.

In another embodiment the invention provides a method of increasing the surface to biovolume ratio of a biofilm comprising administering to the biofilm a composition of the invention.

The invention extends to the use of a composition as described in the specification in a method as described.

In one aspect of the invention there is provided a method of producing a composition of the invention comprising the steps of:

(a) hydrolyzing casein with chymosin to form fragments of casein;

(b) concentrating the fragments of casein;

(c) purifying a fragment of casein; and

(d) adding an amount of cation to the purified casein fragment.

In certain embodiments, the invention provides a composition, wherein the composition is prepared by a method comprising the steps of:

(a) hydrolyzing casein with chymosin to form fragments of casein;

(b) concentrating the fragments of casein;

(c) purifying a fragment of casein; and

(d) adding an amount of cation to the purified casein fragment.

In one embodiment the hydrolyzing of casein in step (a) is by addition of Rennet to a solution of casein.

In one embodiment concentrating the fragments of casein in step (b) is by precipitation of paracasein and the non-glycosylated forms of casein. In another embodiment the concentrating is done by diafiltration.

In one embodiment the purifying of the fragments of casein in step (c) is by separation (e.g. using HPLC), centrifugation or filtration.

In one embodiment the adding of a cation to the purified casein fragment in step (d) is by neutralization and spray (freeze) drying of the supernatant (filtrate) or neutralization, addition of cation ions, preferably zinc, and spray (freeze) drying of supematant (filtrate).

Whilst the concept of conservative substitution is well understood by the person skilled in the art, for the sake of clarity conservative substitutions are those set out below.

Gly, Ala, Val; Ile, Leu, Met;

Asp,. Glu, phosphorylated Ser;

Asn, Gln;

Ser, Thr;

Lys, Arg, His;

Phe, Tyr, Trp, His; and

Pro, Na-alkalamino acids.

As used herein, except where the context requires otherwise, the term “comprise” and variations of the term, such as “comprising”, “comprises” and “comprised”, are not intended to exclude further additives, components, integers or steps.

### BRIEF DESCRIPTION OF THE DRAWINGS

FIG. 1 shows five representative 3-dimensional confocal microscopy images of 16 h S. mutans biofilms showing the effect of a single treatment with KCG (purified κ-casein glycopeptide fragment 106 to 169), KCGPZ(f106-169) (antibiofilm zinc glycopeptide), zinc (20 mM) or Chlorhexidine (0.1%).

FIG. 2 shows the effect of KCGPZ(f106-169) treatment from day 18 to 25 (box) on viability of individual sₚecies of a polymicrobial biofilm. Each CFU count indicated in the graph is the average of 3 plugs sampled. Plugs on each pan was sampled on day 6, 11, 12 & 13 (before KCGPZ(f106-169 treatment), day 19, 20, 21 & 25 (KCGPZ(f106-169 treatment from day 18 to 25) and day 26, 27, 28;32 & 33 (after KCGPZ(f106-169 treatment). CFUs were determined by cultured analysis on selective agar. Total bacteria (▪), A. naeslundii (), V. dispar and F. nucleatum (▴), L. casei (◯), S. mutans (♦), S. sanguis (□).

FIG. 3. The effect of KCGP(f106-169) and KCP(f106-169) on E. faecalis biofilm formation determined after 24 h incubation in a static assay. □=Positive control, [symbol] =Negative control, [symbol] =10 mg/mL NaOCl, ▪=KCGP(f106-169) or KCP(f106-169); * statistically significant difference in comparison to positive and negative controls (p<0.05).

FIG. 4 shows a .RP-HPLC chromatogram of KCGP(f106-169) showing the separation of the glycosylated and nonglycosylated forms of κ-casein(106-169). The glycosylated forms eluted between 18 to 23 min. Nonglycosylated κ-caseinA(106-169) eluted at 27 min and nonglycosylated κ-caseinB(106-169) eluted at 30.9 min. Peptides were identified by mass spectrometric analysis as previously described (Dashper et al. 2005).

FIG. 5 shows representative CLSM images of 16 h S. mutans biofilms stained with BacLight Live/Dead stain showing the effects of KCG, KCGPZ(f106-169) and chlorhexidine treatments. The left images show the staining of cells with propidium iodide which is impermeable to intact cell membranes. The central images are the Syto9 staining that detects all cells and the images on the right show the combination of both propidium iodide and Syto9 staining.

### DETAILED DESCRIPTION OF THE EMBODIMENTS

The present inventors have found that a peptide that has an amino acid sequence of, or functionally similar to, a casein or fragment of a casein, in particular κ-casein, that has at least one amino acid that has been glycosylated can prevent, inhibit or reduce a measurable parameter of a biofilm. In addition, the present inventors have found that a composition comprising a divalent cation and a peptide, wherein the peptide has at least one amino acid that has been glycosylated, preferably at least one amino acid that has been phosphorylated and has an amino acid sequence of a casein or fragment of a casein can prevent, inhibit or reduce a measurable parameter of a biofilm. Preferably, the casein is κ-casein (kappa-casein). The casein may be of bovine origin however casein derived from other animals, for example, goats or sheep is also contemplated. The peptide may .be produced synthetically. In addition, the peptide may be derived from a genetic variant of casein or κ-casein can have antibiofilm properties.

The present invention also provides a composition that reduces the proportion of acidogenic bacteria in a biofilm relative to non-acidogeriic bacteria in the biofilm. The acidogenic bacteria may be Streptococcus mutans.

Previous molecular modeling and Plowman et al. 1997 suggested non-glycosylated Kappa-casein peptides form amphipathic helices and this secondary structure is thought to be important for the antibacterial effect of the peptide. Modelling also suggested that glycosylation would disrupt this helical structure and then one would predict the antibacterial activity would be lost. Unexpectedly, the results herein indicate that the glycosylation promotes the antibiofilm effect.

As used herein, an “antibiofilm” composition, agent or peptide or an “antibiofilm” characteristic of a composition, agent or peptide refers to the ability to prevent, inhibit or reduce a measurable parameter of a biofilm. Non-limiting examples of measurable parameters of a biofilm may be total biomass, average thickness, surface to biovolume ratio, roughness coefficient or bacterial composition and their viability of the biofilm. The composition, agent or peptide does not necessarily affect a measurable parameter of a biofilm by reducing the viability of the cells within the biofilm.

“Bactericidal” is used herein to describe the property of a composition, agent, compound, peptidomimetic or peptide that directly reduces the viability of a bacterium regardless of whether it is in a biofilm or planktonic state.

“Biofilm disrupting activity” is used herein to describe the property of a composition, agent, compound, peptidomimetic or peptide that causes the release of live and/or dead bacteria from the biofilm. The composition, agent, compound, peptidomimetic or peptide may also but not necessarily, reduce the viability of a bacterium in a biofilm or destroy the extracellular mucous matrix. “Release” of bacteria from the biofilm includes increasing the number of bacteria in a biofilm to adopt a planktonic state and increasing the susceptibility of a bacterium in a biofilm to bactericidal agents.

Accordingly, without being bound by any theory, or mode of action, it is believed that non-glycosylated peptides exhibit antibacterial Or antimicrobial effects by reducing the viability of bacteria whereas glycosylated peptides of the invention do not reduce the viability of bacteria in a biofilm but instead exhibit biofilm disrupting activity and cause the bacterial cells to be released from the biofilm. In certain embodiments the glycosylated peptides may cause more of the bacteria in a biofilm to adopt a planktonic state. In other embodiments, the peptides or compositions of the invention may inhibit or reduce the formation of a biofilm. In certain embodiments, the peptides or compositions of the invention may inhibit or reduce biofilm growth. In other embodiments, the peptides or compositions of the invention may inhibit or reduce any characteristic that a biofilm exhibits which initiates or promotes a disease or condition in a subject. In certain embodiments, the peptides or compositions may inhibit or reduce any characteristic that a biofilm exhibits which initiates or promotes a disease or condition in a subject, without killing the bacteria in the biofilm.

A peptide or peptidomimetic that is “functionally similar” to casein or a fragment of casein may have a different structure, i.e. amino acid sequence, length or post-translational modification, but still retains a function of casein or a fragment of casein. Functionally similar peptides or peptidomimetics can be determined by shortening the amino acid sequence, for example using an exopeptidase, or by sythesizing amino acid sequences of shorter length, and then testing for an antibiofilm property.

A ‘peptidomimetic’ is a synthetic chemical compound that has substantially the same structure and/or functional characteristics of a peptide of the invention, the latter being described further herein. Typically, a peptidomimetic has the same or similar structure as a peptide of the invention, for example the same or similar sequence of a peptide derived from casein and is glycosylated. A peptidomimetic generally contains at least one residue that is not naturally synthesised. Non-natural components of peptidomimetic compounds may be according to one or more of: a) residue linkage groups other than the natural amide bond (‘peptide bond’) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

‘Planktonic bacteria’ are bacteria that are suspended or growing in a fluid environment as opposed to those attached to a surface.

A “therapeutically effective amount” as used herein refers to an amount effective, at dosages and for periods of time necessary, to achieve the desired therapeutic result. A desired therapeutic result includes reduction or inhibition in the severity, incidence, progression or risk of developing a disease or condition, or symptom related thereto, wherein the disease or condition includes biofilm, dental plaque, gingivitis, periodontitis, dental caries, oral mucositis, dry mouth or xerostomia. A therapeutically effective amount of a compound, peptide, peptidomimetic or composition of the invention may be determined by a person skilled in the art and may vary according to factors such as the disease state, including severity and stage of progression, age, sex, and weight of the individual, and the ability of a compound, peptide, peptidomimetic or composition of the invention to elicit a desired response in an individual. A therapeutically effective amount is also one in which any toxic or detrimental effects of a compound, peptide, peptidomimetic or composition of the invention are outweighed by the therapeutically beneficial effects.

κ-casein can be glycosylated on various residues including the N-terminus, threonine 121, threonine 131, threonine 133, threonine 136 (not in bovine variant B); threonine 142 and threonine 165 (numbering as per bovine x-casein A (SEQ ID NO: 11). Several glycan forms may be attached to the relevant residue including tetrasaccharides, for example NeuAc(α2-3)Gal(β1-3)[NeuAc(α2-6)]GalNAc, monosaccharides, for example GalNAc, disaccharides, for example Gal(β1-3)GalNAc and trisaccharides for example NeuAc(α2-3)Gal(β1-3)GalNAc or Gal(β1-3)[NeuAc(α2-6)]GalNAc. One preferred embodiment is a glycan including N-acetyl neuraminic acid.

The peptide in a composition of the invention may be phosphorylated on at least one amino acid. The amino acid which may be phosphorylated is serine 127 and/or serine 149 (numbering as per bovine κ-casein A SEQ ID NO: 11). A peptide may be phosphorylated on any other amino acid capable of being phosphorylated either in vivo or in vitro by a kinase.

As used herein, reference to a “peptide that is glycosylated”, “glycopeptide” or “glycosylated peptide” includes a peptide that contains at least one glycan molecule per peptide molecule. In one embodiment, the glycosylation state of a peptide of the invention is that of the most common form of κ-casein (106-169) when derived from bovine κ-casein using standard separation techniques. In another embodiment the glycosylation, state of a peptide of the invention is that of a peptide purified by standard processes of extracting κ-casein from bovine milk.

To illustrate these modifications, a number of genetic variants of bovine casein are known as follows:

κ-Caseins
1. κ-Casein X^{a}-2P (genetic variants-A, B, C, E, F¹, F², G¹, G², H, I and J)
2. κ-Casein X^{a}-2P (f106-169) (genetic variants-A, B, E, F¹, F², G¹, G² and J)
3. κ-Casein X^{a}-2P (f117-169) (genetic variants-A, B, E, F¹, F², G¹, G² and J)
a. X indicates a genetic variant, where the genetic variant may be any one of the variants stated in the following brackets.

The above nomenclature describes known variants of casein, for example, κ-casein B-2P (f106-169) indicates that the protein is part of the κ-family of casein, is the B genetic variant, contains two amino acid residues that are phosphorylated and is a fragment of the κ-casein protein from residue 106 to residue 169. It is known which amino acids are susceptible to phosphorylation in these proteins. The nomenclature and further description of casein variants is described in Farrell et al: Nomenclature of Proteins of Cow's Milk—Sixth Revision. Journal of Dairy Science (2004)87:1641-1674. Accordingly, the fragments of these sequences that correspond to the amino acid sequences of SEQ ID NO: 1. to 10 are variants, and form part of the invention disclosed. κ-casein from other species is also within the scope of the invention, non-limiting examples of species from which peptides of the invention may be derived are humans, cows, goats and sheep. For example, peptides of the invention may be derived from bovine κ-casein A (SEQ ID NO: 11) or bovine κ-casein B (SEQ ID NO: 12).

Bovine κ-casein A
(SEQ ID NO: 11)
¹QEQNQEQPIRCEKDERFFSDKIAKYIPIQYVLSRYPSYGLNYYQQKPVALINNQFLPY1P
YYAKPAAVRSPAQILQWQVLSNTVPAKSCQAQPTTMARHPHPHLSFMAIPPKKNQDK
TEIPTINTIASGEPTSTPTTEAVESTVATLEDSPEVIESPPEINTVQVTSTAV¹⁶⁹
Bovine κ-casein B (amino acid substitutions relative to
bovine κ-casein A are underlined)
(SEQ ID NO: 12)
¹QEQNQEQPIRCEKDERFFSDKIAKYIPIQYVLSRYPSYGLNYYQQKPVALINNQFLPYP
YYAKPAAVRSPAQILQWQVLSNTVPAKSCQAQPTTMARHPHPHLSFMAIPPKKNQDK
TEIPTINTIASGEPTSTPTIEAVESTVATLEASPEVIESPPEINTVQVTSTAV¹⁶⁹

As used herein:

KCP(f106-169)=κ-casein peptide fragment 106 to 169

KCGP(f106-169)=κ-casein glycopeptide preparation fragment 106 to 169 prepared as per Example 1.

KCGPZ(f106-169)=κ-casein glycopeptide preparation fragment 106 to 169 +Zn2+(which may also be referred to as a zinc complex of KCGP) prepared as per Example 1.

KCG. or KCG(f106-169)=purified κ-casein glycopeptide fragment 106 to 169 prepared as per Example 1.

The efficacy of KCGPZ(f106-169) has been shown herein on a multispecies oral biofilm cultured in a constant-depth film fermenter (CDFF) and on S. mutans biofilms cultured . in a flow cell model with Confocal Scanning Laser Microscopy (CSLM) analysis. The efficacy and action of KCGPZ(f106-169) was compared with Chlorhexidine and zinc ions alone. KCGPZ(f106-169) was more effective against 16 h S. mutans biofilms than Chlorhexidine and it resulted in a much higher surface area to volume ratio than obtained with either Chlorhexidine or zinc treatment. Overall, KCGPZ(f106-169) has shown its efficacy against the acidogenic S. mutans 16 h biofilms and suppresses the recovery of these biofilms. Similar observations of KCGPZ(f106-169) were shown in the suppression of the acidogenic species when it was tested on a multispecies oral biofilm cultured in a CDFF.

In certain embodiments of the invention a peptidomimetic based on a peptide of the invention may be used in a composition or method of treatment described.

The invention also includes functional fragments of the amino acid sequences of SEQ ID NO: 1 to 10. A functional fragment is an amino acid sequence that is shorter or longer than the amino acid sequences corresponding to any one of SEQ ID NOs: 1 to 10 but still retains the function of the corresponding amino acid sequences to SEQ ID NO: 1 to 10. A functional fragment can be easily determined by shortening or lengthening the amino acid sequence, for example using an exopeptidase, or by synthesizing amino acid sequences. of shorter or longer length, and then testing for any activity, for example, antibiofilm activity.

Also within the scope of the invention are variants of the amino acid sequences of SEQ ID NO: 1 to 10 corresponding to orthologous or paralogous sequences.

The peptide, peptidomimetic or composition of the invention may be administered directly to a tooth or gums, for example at disto-buccal, mid-buccal, mesio-buccal, mesio-palatal, mid-palatal and disto-palatal and disto-lingual, mid-lingual and mesio-lingual site, of the subject in need of treatment or prevention of periodontal disease. Topical administration of the composition of the invention is preferred, however it will be appreciated by a person skilled in the art that a compound, peptide, peptidomimetic or composition may also be administered parenterally, e.g, by injection intravenously, intraperitoneally, intramuscularly, intrathecally or subcutaneously.

Although the invention finds application in humans, the invention is also useful for veterinary purposes. The invention is useful for domestic animals such as cattle, sheep, horses and poultry; for companion animals such as cats and dogs; and for zoo animals.

A subject in need of treatment may be one which exhibits subclinical or clinical symptoms of dental caries, gingivitis, periodontitis, oral mucositis, periodontal disease, dry mouth or xerostomia. In one embodiment, the subject in need of treatment has been identified as exhibiting subclinical or clinical symptoms of dental caries, gingivitis, periodontitis, oral mucositis, periodontal disease, dry mouth or xerostomia.

Subclinical or clinical manifestations of periodontal disease include acute or chronic inflammation of the gingiva. The hallmarks of acute inflammation may be present including an increased movement of plasma and leukocytes from the blood into the injured tissues. Clinical signs of acute infection of the gingiva may also be present including rubor (redness), calor (increased heat), tumor (swelling), dolor (pain), and functio laesa (loss of function). Chronic inflammation may be characterised by leukocyte cell (monocytes, macrophages, lymphocytes, plasma cells) infiltration. Tissue and bone loss may be observed. A subject in need of treatment may also be characterised by having an increased level of P. gingivalis bacteria present at a periodontal site, above a normal range observed in individuals without periodontal disease.

The route of administration may depend on a number of factors including the nature of the peptide, peptidomimetic or composition to be administered and the severity of the subject's condition. It is understood that the frequency of administration of a compound, peptide, peptidomimetic or composition of the invention and the amount of compound, peptide, peptidomimetic or composition of the invention administered may be varied from subject to subject depending on, amongst other things, the stage of periodontal disease initiation or progression in the subject. The frequency of administration may be determined by a clinician.

It is also contemplated that any disease, condition or syndrome that is a consequence of or associated with a biofilm, may be prevented or treated by a peptide, peptidomimetic or composition of the invention. In addition, a symptom of a disease, condition or syndrome that is a consequence of or associated with a biofilm may be reduced in severity or incidence by a peptide, peptidomimetic or composition of the invention. Furthermore, other diseases, conditions or syndromes that are a consequence of or associated with periodontal disease may also be treated or the risk of developing these diseases, conditions or syndromes may be reduced. For example, periodontal disease may increase the risk of an individual developing cardiovascular disease. This increase risk of developing cardiovascular disease may be reduced by treating periodontal disease by administering a peptide, peptidomimetic or composition of the invention to an individual with periodontal disease.

As stated herein, a peptide, peptidomimetic or composition of the invention may be used as an oral lubricant, saliva substitute or artificial saliva. An oral lubricant is capable of moistening the mouth and lubricating the surfaces of the oral cavity. An oral lubricant may act in addition to saliva, synergistically with saliva or in the absence of saliva as a saliva substitute. An oral lubricant is particularly useful for patients with substantially or completely defective (or absent) salivary glands and therefore who produce little or no saliva. In one embodiment, a peptide, peptidomimetic or composition of the invention may further include a saliva stimulant, such as pilocarpine, which stimulates the saliva gland to generate more saliva. Patients in need of an oral lubricant, saliva substitute or artificial saliva may be those suffering from dry mouth or xerostomia as a result of Sjögren's syndrome, poorly controlled diabetes, Lambert-Eaton syndrome, radiotherapy or chemotherapy.

Typically, any component that is found in natural saliva may also be a component of the composition of the invention. In one aspect, saliva constituents are selected from the group consisting of sodium, potassium, chlorides, fluorides, phosphates, bicarbonates, oxygen, carbon dioxide, urea, enzymes such as ptyalin, maltase and amylase, and proteins such as mucin, globulin, albumen and statherin.

The functions of the peptide, peptidomimetic or composition used as a oral lubricant, saliva substitute or as artificial saliva include those of natural saliva; such as: wash away food debris and plaque from the teeth to help prevent caries; limit the growth of bacteria that cause tooth decay, mouth odour (halitosis), and other mouth infections; bathe the teeth and supply minerals such as calcium and phosphate that allow remineralisation of tooth structure; lubricate foods so that they may be swallowed more easily; moisten the inside of the mouth to make chewing and speaking easier; provide enzymes that aid in digestion and increase enjoyment of foods by aiding in the “tasting” process.

“Percent (%) amino acid sequence identity” or “percent (%) identical” with respect to a peptide or polypeptide sequence, i.e. a peptide of the invention defined herein, is defined as the percentage of amino acid residues in a candidate sequence that are identical with the amino acid residues in the specific peptide or polypeptide sequence, i.e. a peptide of the invention, after aligning the sequences and introducing gaps, if necessary, to achieve the maximum percent sequence identity, and not considering any conservative substitutions as part of the sequence identity.

Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite. (lnvitrogen Corporation, Carlsbad, Calif.). After alignment of amino acid sequences with ClustalW, the percent amino acid-identity can be assessed. A non-limiting example of a software program useful for analysis of ClustalW alignments is GENEDOC™. GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, Calif., USA). When utilizing the ALIGN program for comparing amino acid sequences, a PAM 120 weight residue table, a gap length penalty of 12, and a gap penalty of 4 can be used.“Percent (%) amino acid sequence identity” or “ percent (%) identical” with respect to a peptide or polypeptide sequence, i.e. a peptide of the invention defined herein, is defined as the percentage, of amino acid residues in a candidate sequence that are identical with the amino acid residues in the specific peptide or polypeptide sequence, i.e. a peptide of the invention, after aligning the sequences and introducing gaps, if necessary, to achieve the maximum percent sequence identity, and not considering any conservative substitutions as part of the sequence identity.

In calculating percent identity, typically exact matches are counted. The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. ScI. USA 87:2264, modified as in Karlin and Altschul (1993) Proc. Natl. Acad. ScI. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between ,molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus, can provide data about the sequence conservation of the entire amino acid sequence. The ClustalW algorithm is used in several commercially available DNA/amino acid analysis software packages, such as the ALIGNX module of the Vector NTI Program Suite (lnvitrogen Corporation, Carlsbad, Calif.). After alignment of amino acid sequences with ClustalW, the percent amino acid identity can be assessed. A non-limiting example of a software program useful for analysis of ClustalW alignments is GENEDOC™. GENEDOC™ allows assessment of amino acid (or DNA) similarity and identity between multiple. proteins. Another non-limiting example of a mathematical algorithm utilized for the comparison of sequences is the algorithm of Myers and Miller . (1988) CABIOS 4:11-17. Such an algorithm is incorporated into the ALIGN program (version 2.0), which is part of ,the GCG Wisconsin Genetics Software Package, Version 10 (available from Accelrys, Inc., 9685 Scranton Rd., San Diego, Calif., USA). When utilizing the ALIGN program for comparing amino acid sequences, a PAM 120 weight residue table, a gap length penalty of 12, and a gap penalty of 4 can be used.

An oral composition of this invention which contains the above-mentioned pharmaceutical composition may be prepared and used in various forms applicable to the mouth such as dentifrice including toothpastes, toothpowders and liquid dentifrices, mouthwashes, oral lubricant, saliva substitute, artificial saliva, troches, chewing gums, dental pastes, gingival massage creams, gargle tablets, dairy products and other foodstuffs. An oral composition according to this invention may further include additional well known ingredients depending on the type and form of a particular oral composition.

Optionally, the composition may further include one or more antibiotics that are toxic to or inhibit the growth of Gram negative anaerobic bacteria. Potentially any bacteriostatic or bactericidal antibiotic may be used in a composition of the invention. Preferably, suitable antibiotics include amoxicillin, tetracycline, doxycycline or metronidazole.

In certain preferred forms of the invention the oral composition may be substantially liquid in character, such as a mouthwash or rinse. In such a preparation the vehicle is typically a water-alcohol mixture desirably including a humectant as described below. Generally, the weight ratio of water to alcohol is in the range of from about 1:1 to about 20:1. The total amount of water-alcohol mixture in this type of preparation is typically in the range of from about 70 to about 99.9% by weight of the preparation. The alcohol is typically ethanol or isopropanol. Ethanol is preferred.

The pH of such liquid and other preparations of the invention is generally in the range of from about 5 to about 9 and typically from about 5.0 to 7.0. The pH can be controlled with acid (e.g. citric acid or benzoic acid) or base (e.g. sodium hydroxide) or buffered (as with sodium citrate, benzoate, carbonate, or bicarbonate, disodium hydrogen phosphate, sodium dihydrogen phosphate, etc).

In other desirable forms of this invention, the composition may be substantially solid or pasty in character, such as toothpowder, a dental tablet or a toothpaste (dental cream) or gel dentifrice. The vehicle of such solid or pasty oral preparations generally contains dentally acceptable polishing material.

In a toothpaste, the liquid vehicle may comprise water and humectant typically in an amount ranging from about 10% to about 80% by weight of the preparation. Glycerine, propylene glycol, sorbitol and polypropylene glycol exemplify suitable humectants/carriers. Also advantageous are liquid mixtures of water, glycerine and sorbitol. In clear gels where the refractive index is an important consideration, about 2.5-30% w/w of water, 0 to about 70% w/w of glycerine and about 20-80% w/w of sorbitol are preferably employed.

Toothpaste, creams and gels typically contain a natural or synthetic thickener or gelling agent in proportions of about 0.1 to about 10, preferably about 0.5 to about 5% w/w. A suitable thickener is synthetic hectorite, a synthetic colloidal magnesium alkali metal silicate complex clay available for example as Laponite (e.g. CP, SP 2002, D) marketed by Laporte Industries Limited. Laponite D is, approximately by weight 58.00% SiO₂, 25.40% MgO, 3.05% Na₂O, 0.98% Li₂O, and some water and trace metals. Its true specific gravity is 2.53 and it has an apparent bulk density of 1.0 g/ml at 8% moisture.

Other suitable thickeners include Irish moss, iota carrageenan, gum tragacanth, starch, polyvinylpyrrolidone, hydroxyethylpropylcellulose, hydroxybutyl methyl cellulose, hydroxypropyl methyl cellulose, hydroxyethyl cellulose (e.g. available as Natrosol), sodium carboxymethyl cellulose, and colloidal silica such as finely ground Syloid (e.g. 244). Solubilizing agents may also be included such as humectant polyols such propylene glycol, dipropylene glycol and hexylene glycol, cellosolves such as methyl cellosolve and ethyl cellosolve, vegetable oils and waxes containing at least about 12 carbons in a straight chain such as olive oil, castor oil and petrolatum and, esters such as amyl acetate, ethyl acetate and benzyl benzoate.

It will be understood that, as is conventional, the oral preparations will usually be sold or otherwise distributed in suitable labelled packages. Thus, a bottle of mouth rinse will have a label describing it, in substance, as a mouth rinse or mouthwash and having directions for its use; and a toothpaste, cream or gel will usually be in a collapsible tube, typically aluminium, lined lead or plastic, or other squeeze, pump or pressurized dispenser for metering out the contents, having a label describing it, in substance, as a toothpaste, gel or dental cream.

Organic surface-active agents may be used in the compositions of the present invention to achieve increased therapeutic or prophylactic action, assist in achieving thorough and complete dispersion of the active agent throughout the oral cavity, and render the instant compositions more cosmetically acceptable. The organic surface-active material is preferably anionic, non-ionic or ampholytic in nature and preferably does not interact with the active agent. It is preferred to employ as the surface-active agent a detersive material which imparts to the composition detersive and foaming properties. Suitable examples of anionic surfactants are water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkylsulfo-acetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine which should be substantially free from soap or similar higher fatty acid material. The use of these sarconite compounds in the oral compositions of the present invention is particularly advantageous since these materials exhibit a prolonged marked effect in the inhibition of acid formation in the oral cavity due to carbohydrates breakdown in addition to exerting some reduction in the solubility of tooth enamel in acid solutions. Examples of water-soluble nOn-ionic surfactants suitable for use are condensation products of ethylene oxide with various reactive hydrogen-containing compounds reactive therewith having long hydrophobic chains (e.g. aliphatic chains of about 12 to 20 carbon atoms), which condensation products (“ethoxamers”) contain hydrophilic polyoxyethylene moieties, such as condensation products of poly (ethylene oxide) with fatty acids, fatty alcohols, fatty amides, polyhydric alcohols (e.g. sorbitan monostearate) and polypropyleneoxide (e.g. Pluronic materials).

The surface active agent is typically present in amount of about 0.1-5% by weight. It is noteworthy, that the surface active agent may assist in the dissolving of the active agent of the invention and thereby diminish the amount of solubilizing humectant needed.

Various other materials may be incorporated in the oral preparations of this invention such as whitening agents, preservatives, silicones, chlorophyll compounds and/or ammoniated material such as urea, diammonium phosphate, and mixtures thereof.

These adjuvants, where present, are incorporated in the preparations in amounts which do not substantially adversely affect the properties and characteristics desired.

Any suitable flavouring or sweetening material may also be employed. Examples of suitable flavouring constituents are flavouring oils, e.g. oil of spearmint, peppermint, wintergreen, sassafras, clove, sage, eucalyptus, marjoram, cinnamon, lemon, and orange, and methyl salicylate. Suitable sweetening agents include sucrose, lactose, maltose, sorbitol, xylitol, sodium cyclamate, perillartine, AMP (aspartyl phenylalanine, methyl ester), saccharine, and the like. Suitably, flavour and sweetening agents may each or together comprise from about 0.1% to 5% more of the preparation.

The compositions of the invention can also be incorporated in lozenges, or in chewing gum or other products, e.g. by stirring into a warm gum base or coating the outer surface of a gum base, illustrative of which are jelutong, rubber latex, vinylite resins, etc., desirably with conventional plasticizers or softeners, sugar or other sweeteners or such as glucose, sorbitol and the like.

In a further aspect, the present invention provides a kit of parts including (a) a composition of the invention and (b) a pharmaceutically acceptable carrier. Desirably, the kit further includes instructions for their use for the treatment or prevention of periodontal disease in a patent in need of such treatment.

Compositions intended for oral use may be prepared according to any method known in the art for the manufacture of pharmaceutical compositions and such compositions may contain one or more agents selected from the group consisting of sweetening agents, flavouring agents, colouring agents, and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may;also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

The aqueous suspensions may also contain one or more preservatives, for example benzoates, such as ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

In order that the nature of the invention may be more readily understood, forms of the invention will now be described with reference to the following non-limiting examples. In these examples “KCGPZ(f106-169) ” or “antibiofilm zinc glycopeptide” refers to a composition having kappa (κ) casein glycopeptide fragment 106 to 169+Zn2+ (or Zinc complex) prepared as illustrated in Example 1 and used in studies described in Examples 2 and 3. “KCG” or “KCG(f106-169)” refers to purified x-casein glycopeptide fragment 106 to 169 prepared as per Example 1 and used in studies described in the Examples. “KCGP(f106-169)” refers to ic-casein glycopeptide preparation fragment 106 to 169 prepared as per Example 1 and used in studies described in the Examples. “KCP(f106-169)” refers to non-glycosylated κ-casein peptide fragment 106 to 169 and used in studies described in the Examples.

It will be understood that the invention disclosed and defined in this specification extends to all alternative combinations of two or more of the individual features mentioned or evident from the text or drawings. All of these different combinations constitute various alternative aspects of the invention.

### EXAMPLE 1

KCGP(f106-169) and KCGPZ(f106-169) Preparation Casein-HCl was dissolved in deionized water at 50° C. to a final concentration of 21.5 g/L, the pH was maintained at 8.0 by the addition of 1 M NaOH. The temperature was then lowered to 37° C. and the pH adjusted to 6.3 by addition of 1 M HCl to avoid precipitation of casein. To hydrolyze the casein, Rennet (90% chymosin; EC 3.4.23.4; 145 international Milk clotting units [IMCU]/mL; single strength; Chr. Hanson) was added to a final concentration of 1.2 IMCU/g casein and the solution was stirred at 37° C. for 1 h. Hydrolysis was stopped by the addition of trichloroacetic acid to a final concentration of 4%, and the precipitated proteins were pelleted by centrifugation (5,000 g, 15 min, 4° C.). The supernatant containing κ-casein(106-169) caseinomacropeptide (CMP) was concentrated and washed with H₂O using diafiltration with a 3,000-Da cutoff membrane (S10Y3, Amicon/Millipore) to produce KCGP(f106-169). The retentate was analyzed by high-pressure liquid chromatography (HPLC) and mass spectrometric analysis. KCGPZ(f106-169) was prepared by adding 20 mM ZnCl₂ to a 10 mg/mL of the KCGP(f106-169) preparation. This solution was the KCGPZ(f106-169) solution used in the biofilm assays.

Purification of glycosylated κ-casein(106-169)—KCG. KCGP(f106-169), prepared as described above, was dissolved in 0.1% (v/v) trifluoroacetic acid (TFA) in water (solvent A) and applied to a C18 semi-preparative RP column (250×10 mm, Vydac) installed in an Agilent 110 HPLC system. The peptides were eluted using 5% solvent B at an initial flow rate 0.1 mL/min which increased to 3.5 mL/min in the first min. The gradient increased to 15% solvent B in the next min, followed by a gradient of 15-30% solvent B for 10 min, a gradient of 30-48% solvent B for 24 min, a gradient of 48-100% solvent for 2 min. Solvent B contained 80% acetonitrile with 20% water containing 0.085% (v/v) TFA. The eluant was monitored usin_{g} a primary wavelength of 215 nm. The fractions collected between 18 to 23 min of elution were collected, pooled and freeze dried. The calculated yield of KCG was 24% of KCGP(f106-169).

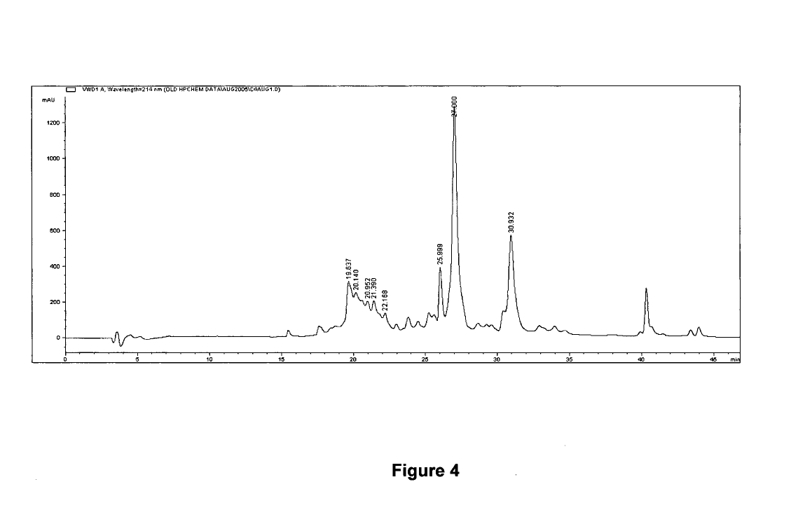

Analysis of protein preparations. KCGP(f106-169) when prepared as described above contained 43% nonglycosylated κ-casein(106-169) and 24% glycosylated κ-casein(106-169). This material was further purified by reversed phase HPLC to separate the glycosylated forms of κ-casein(106-169) from the nonglycosylated forms (FIG. 4). The glycosylated forms of κ-casein(106-169) eluting between 18-23 min were collected and herein referred to as KCG.

### EXAMPLE 2

Flowcell culture and CSLM analysis of monospecific biofilm The biofilm culture of S. mutans in flow cells was similar to that described by Wen et al. (2006) with several modifications. A 3-channel flow cell system (Stovall Life Science, Greensboro, N.C., USA) was modified with stopcocks for inoculation, testing and staining of the bacterial biofilms. All parts were assembled and 0.5% sodium hypochlorite was pumped in and left overnight. Sterile water (200 mL) was then used to flush the system prior to the addition of growth medium. The system was inoculated with 1 mL of an exponentially growing S. mutans Ingbritt culture diluted to a cell density of 1×10⁷ cells/mL. The system was incubated for 1 h prior to constant flow (0.2 mL/min) of 25% ASM (ASM; 2.5 g/L type II porcine gastric mucin, 2.0 g/L bacteriological peptone, 2.0 g/L tryptone, 1.0 g/L yeast extract, 0.35 g/L NaCl, 0.2 g/L KCl, 0.2 g/L CaCl₂ and 1 mg/L haemin, pH 7.0) supplemented with 2.5 mM DTT and 0.5 g/L sucrose.. After 16 h, 1 mL of purified glycosylated x-casein(106-169) (KCG); KCGP(f106-169); KCGPZ(f106-169) (KCGPZ(f106-169); 10 mg/mL KCGP(f106-169) and 20 mM ZnCl₂); 20 mM ZnCl₂; 0.1% chlorhexidine digluconate or sterile water was injected into each channel of the system and incubated for 10 min, the flow of 25% ASM was then resumed for 10 min prior to staining. For determination of the effects of treatments on early stage biofilms the system was incubated for 6 h after inoculation, treated with 1 mL of test solutions for 10 min and the flow of 25% ASM resumed for another 16 h to observe recovery of the biofilm. BacLight LIVE/DEAD stain (Molecular Probes) was then used to stain the biofilm in situ.

Confocal laser scanning microscopy (CLSM) of the bacterial biofilms was carried out on a Meta 510 Confocal Microscope with an inverted stage (Zeiss). Horizontal (xy) optodigital sections, each 2 μm thick over the entire thickness of the biofilm (z) were imaged using a 63× objective at 512×512 pixel (0.28 μm per pixel), with each frame at 143.86 μm (x)×143.86 μm (y). To determine reproducibility across the biofilm 5 images at random positions were obtained at wavelengths of 488 nm and 568 nm for each channel and 3 biological replicates were used. All images obtained were analysed using Comstat software (Heydorn et al., 2000).

Data treatment and statistical analyses. The biometric data were analyzed using a two-factor analysis of variance (ANOVA) model with treatment included as a fixed factor and experiment as a random blocking factor to investigate the effect of KCGP(f106-169), KCG, KCGPZ(f106-169), Zinc and chlorhexidine on established biofilms. If treatment differences were significant, post hoc comparisons of treatment differences were performed using the Tukey posttest. Model fits were checked by residual plots, normality was investigated using normal probability plots and the Kolmogorov-Smirnov test and homogeneity of error variances was tested using Levene's test. When the error variances were heterogeneous a natural logarithm transformation of the data was used to stabilize the treatment group variances. Treatments were then compared using ANOVA on the transformed data. The biometric data obtained from the experiments investigating the effect of KCGPZ(f106-169) on 6 h S. mutans biofilms was analysed using a Paired Samples T-Test. All analyses were conducted using SPSS statistical software (version 17.0, SPSS Inc., Chicago, Ill.).

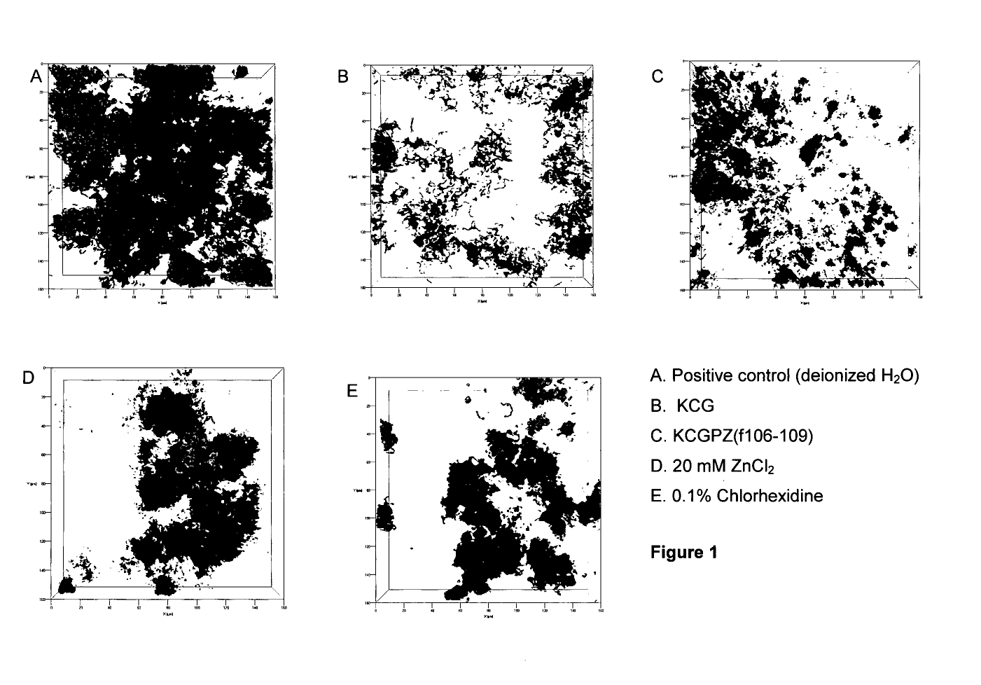

When grown in a flow cell for 16 h with a dilute artificial saliva medium containing mucin S. mutans produced a dense, structured biofilm with an average thickness of 7.37 μm and a biovolunne of 3.88 μm³ per μm² of substratum (Table 1). All treatments had obvious impacts on the S. mutans biofilm structure, including average thickness and biovolume (FIG. 1 and Table 1). A single treatment with purified KCG at concentrations of 2.4 and 10 mg/mL resulted in a dose dependent disruption of the S. mutans biofilms with significant decreases in the average thickness of the biofilms of 69 and 86%, respectively and decreases in the biovolume by 59 and 78%, respectively (Table 1). Treatment of these biofilms with 10 mg/mL of KCGP(f106-169), that contained 2.4 mg/mL KCG, produced the same results as 2.4 mg/mL purified KCG (Table 1). The roughness coefficient increased markedly with KCG (10 mg/mL) and KCGPZ(f106-169) treatments indicating significant disruption of the biofilm structure.

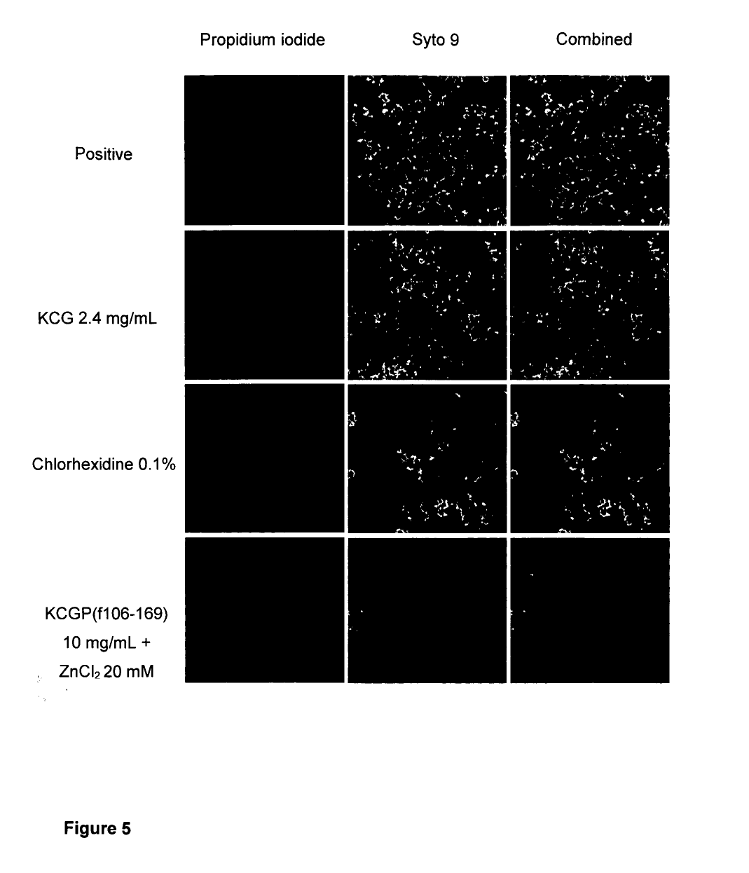

Treatment of S. mutans biofilms with KCGPZ(f106-169), a combination of 10 mg/mL KCGP(f106-169) and 20 mM ZnCl₂, produced significantly better disruption of the biofilms than treatment with 10 mg/mL KCGP(f106-169) or 20 mM ZnCl₂ alone (Table 1). Chlorhexidine treatment reduced total biomass and average thickness by 53% and 59%, respectively and produced results that were not significantly different to those produced by ZnCl₂, 2.4 mg/mL purified KCG or 10 mg/mL KCGP(f106-169) (Table 1). Biofilms treated with chlorhexidine displayed a qualitatively higher number of dead S. mutans cells compared with KCG or KCGPZ(f106-169), as determined by staining with propidium iodide (FIG. 5). In other words, more cells displayed staining with propidium iodide after treatment with chlorhexidine (first column third panel from top in FIG. 5) than either KCG (first column second panel from top in FIG. 5) or KCGPZ(f106-169) (first column fourth panel from top in FIG. 5). As stated above, in FIG. 5, the left images (first column) show the staining of cells with propidium iodide which is impermeable to intact cell membranes. The central images (middle column) are the Syto9 staining that detects all cells and the images on the right (right column) show the combination of both propidium iodide and Syto9 staining.

TABLE 1
Biometric parameters of S. mutans biofilms.

| | | | | | Roughness |
|---|---|---|---|---|---|
| | | | | | coefficient |
| | | | Average | Surface to | (dimensionless, |
| | | Biovolume | thickness | biovolume ratio | range: zero- |
| | N | (μm³/μm²) | (μm) | (μm²/μm³) | infinity) |
| Control | 10 | 3.88 ± 0.77 | 7.37 ± 2.60 | 1.99 ± 0.45^{a} | 1.08 ± 0.15 |
| ZnCl₂—20 mM | 3 | 1.86 ± 0.42^{a} | 2.90 ± 0.80^{a} | 2.04 ± 0.61^{ab} | 1.54 ± 0.07^{ab} |
| Chlorhexidine— | 3 | 1.82 ± 0.60^{a} | 3.02 ± 0.66^{a} | 2.36 ± 1.24^{ab} | 1.51 ± 0.12^{a} |
| 0.1% | | | | | |
| KCGP(f106-169)— | 3 | 1.83 ± 0.30^{a} | 2.84 ± 0.87^{a} | 2.32 ± 0.46^{ab} | 1.40 ± 0.13^{a} |
| 10 mg/mL | | | | | |
| KCG—2.4 mg/mL | 3 | 1.58 ± 0.30^{a} | 2.28 ± 0.73^{a} | 2.56 ± 0.27^{ab} | 1.44 ± 0.10^{a} |
| KCG—10 mg/mL | 4 | 0.84 ± 0.09^{b} | 1.00 ± 0.60^{b} | 3.01 ± 0.29^{b} | 1.67 ± 0.06^{b} |
| KCGPZ(f106-169)— | 3 | 0.78 ± 0.08^{b} | 1.17 ± 0.18^{b} | 2.65 ± 0.77^{ab} | 1.65 ± 0.12^{b} |
| 10 mg/mL CMP + | | | | | |
| 20 mM ZnCl₂ | | | | | |

After inoculation the biofilms were cultured for 16 h in flow cell systems and then given a single 10 min treatment with KCG, KCGP(f106-169), KCGPZ(f106-169), ZnCl₂ or chlorhexidine digluconate prior to imaging with CSLM.

Values within columns with the same superscript were not significantly different (p<0.05). As significant heterogeneity in the error variances was found for both total biomass (p<0.001) and average thickness (p<0.001) measurements, the data were log transformed before analysis. The error variances for the log transformed measurements were homogenous and the residuals normally distributed.

When cultured for 6 h in a flow cell S. mutans formed early stage biofilms with an average biovolume of 0.25±0.19 μm³ per μm² of substratum, an average thickness 0.32±0.31 μm, a surface to biovolume ratio of 2.91±0.63 μm²/μm³ and a roughness coefficient of 1.90±0.07. These early stage biofilms were given a single 10 min treatment with either KCGPZ(f106-169) or chlorhexidine and allowed to recover for 16 h, at which time they were imaged by CSLM and the biometric parameters of the biofilms determined. Treatment with KCGPZ(f106-169) resulted in an average reduction of 75% in total biovolume and 73% in average thickness and an increase of surface to biovolume ratio and roughness coefficient compared with the control (Table 2). Chlorhexidine was more effective against these early S. mutans biofilms, substantially reducing the biofilms, which had not recovered after 16 h (Table 2).

TABLE 2
Biometric parameters of S. mutans biofilm
cultured in the flow cell system.

| | | KCGPZ(f106- | |
|---|---|---|---|
| | Control | 169) | Chlorhexidine |
| Biovolume | 4.37 ± 0.95 | 1.10^{a} ± 0.43 | —^{b} |
| (μm³/μm²) | | | |
| Average thickness | 6.28 ± 1.98 | 1.67^{a} ± 0.74 | —^{b} |
| (μm) | | | |
| Surface to biovolume | 1.19 ± 0.19 | 2.91^{a} ± 0.53 | —^{b} |
| ratio (μm²/μm³) | | | |
| Roughness coefficient | 1.08 ± 0.13 | 1.63^{a} ± 0.11 | —^{b} |
| (dimensionless, range: | | | |
| zero-infinity) | | | |

After inoculation the biofilms were cultured for 6 h then treated with 1 mL KCGPZ(f106-169) (10 mg/mL KCGP(f106-169) with 20 mM ZnCl₂) or 0.1% chlorhexidine digluconate for 10 min. Growth medium flow was then resumed and the biofilm allowed to recover for 16 h.
^{a}Significantly different when compared with control using a T-test (p < 0.05).
^{b}No significant amount of biofilms detected after 16 h recovery

### EXAMPLE 3

Multispecies oral biofilms culture. KCGPZ(f106-169) was then tested against a polymicrobial biofilm consisting of six bacterial species which were chosen to represent the major species of supragingival dental plaque, including the opportunistic pathogens that are commonly associated with dental caries initiation and progression. The biofilm bacterial growth media (ASM) was designed to mimic the glycoprotein-rich composition of saliva and a carbohydrate and protein mixture was pulsed into the CDFF four times per day to mimic dietary intake.

To model supragingival plaque six oral bacterial species were Streptococcus sanguis (NCTC 7863), Streptococcus mutans Ingbritt, Actinomyces naeslundii (NCTC 10301), Veillonella dispar (ATCC 17745), Lactobacillus casei (NCDO 161) and Fusobacterium nucleatum (ATCC 10953) and were cultured as a polymicrobial biofilm in a constant-depth film fermentor (CDFF; Cardiff University, Cardiff, United Kingdom; (Dashper et al. 2005; Shu et al., 2003; Wilson, 1996; Wimpenny et al., 1989), at 37° C. under anaerobic conditions which were maintained by a constant flow of 5% CO₂ in N₂ at 1 L/h. The CDFF contained 15 removable polytetrafluoroethylene (PTFE) pans on a circular platform that was rotated at a constant speed of 3 rpm. The biofilms were grown on five plugs in each PTFE pan that were recessed to a depth of 300 pm using an artificial saliva medium (ASM) (pH 7.0) containing 2.5 g/L mucin (porcine, gastric, type II), 2:0 g/L bacteriological peptone, 2.0 g/L tryptone, 1.0 g/L yeast extract, 0.35 g/L NaCl, 0.2 g/L KCl, 0.2 g/L CaCl₂ and 1 mg/L haemin (McBain et al., 2003b; Pratten et al., 1998) at a constant flow rate of 30 mL/h. This was supplemented with a four times daily addition of a mixture of 5 g/L soluble starch, 2 g/L sucrose, 3 g/L casein, 3 g/L bacteriological peptone, 2.5 g/L yeast extract, 4.5 g/L NaCl, 0.2 g/L K₂HPO₄, 0.4 g/L CaCl₂, 0.2 g/L Na HCO₃, for 30 min at a flow rate of 20 mL/h (McBain et al., 2003a; b) to simulate dietary intake. Prior to inoculation, the CDFF plug surfaces were conditioned for 24 h with ASM at a flow rate of 10 mL/h (McBain et al., 2003b). Each bacterial species was grown individually in batch culture in Brain Heart Infusion (37 g/L) for 40 h. A sample of each culture (10 ml) was then mixed gently with an equal amount of ASM and inoculated into the CDFF at a flow rate of 20 mL/h over a 6 h period.

At the indicated sampling time each pan was removed aseptically from the CDFF, three plugs were extracted from the pan and planktonic cells were removed by gentle washing with 100 μL of ASM. Each plug was then placed in 1 mL of medium and vortexed for 60 s to disrupt the biofilm on the plug. The supernatant was then serially diluted from 10¹ to 10⁶ and 100 μL of each dilution was plated onto a range of selective media for colony forming unit (CFU) count. The selective media were Wilkins-Chalgren agar (total anaerobes); Wilkins-Chalgren agar with gram-negative supplement (total gram-negative anaerobes); Cadmium Fluoride Acriflavin Tellurite agar (dental actinomycetes); Mitis-Salivarius agar (Streptococcus spp.); Mitis-Salivarius agar with 0.1 Unit/mL bacitracin (S. mutans); Rogosa agar (total lactobacilli) (McBain et al., 2003b). These agars were incubated at 37° C. in an anaerobic chamber for up to 5 days. Morphologically distinct bacterial colonies were counted and bacteria gram stained to determine purity and cellular morphology. After the biofilm stabilized, 1.67 mL of KCGPZ(f106-169) was added 4 times daily for 7 days, 5 min after each supplement feeding, at a flow rate of 20 mL/h.

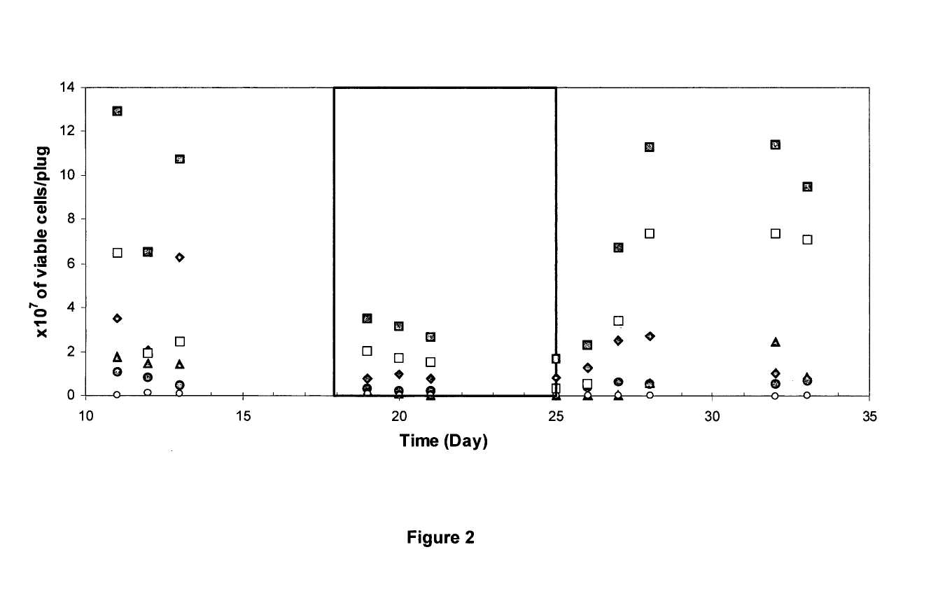

The polymicrobial biofilm consisted of 42% S. mutans, 33% S. sanguis, 7% A. naeslundii, 2% L. casei, 16% both F. nucleatum and V. dispar 13 days after inoculation (Table 3). The first 5 min application of KCGPZ(f106-169) markedly reduced the total bacterial cell count of the biofilm and repeated application over the 7 day test period continued to reduce the bacterial cell count (FIG. 2). At the end of the KCGPZ(f106-169) treatment period the total bacterial cell count was 14% of that ₚrior to treatment. At the end of KCGPZ(f106-169) treatment, there had been a reduction of viable S. mutans, S. sanguis A. naeslundii, L. casei, both F. nucleatum and V. dispar by 87%, 95%, 55%, 51% and 98%, respectively. The total bacterial cell count of the biofilm took ˜3 days to recover from KCGPZ(f106-169) treatment (FIG. 2).

Although the viability of F. nucleatum and V. dispar was reduced by KCGPZ(f106-169) treatment, their proportions as a percentage of the total viable bacterial population, together with A. naeslundii remained unchanged before and after the treatment (Table 3). However KCGPZ(f106-169) treatment significantly suppressed the recovery of S. mutans and lowered its percentage of the total population from 51% to 9%. Similarly, the proportion of the acidogenic L. casei as a percentage of the total bacterial population decreased from 2% to 0.3% whereas the percentage of S. sanguis increased from 21% to 69% of the total population (Table 3). Interestingly, the proportion of streptococci as a whole remained fairly stable before and after treatment.

TABLE 3
Effect of KCGPZ(f106-169) treatment on the species composition (%)
of a polymicrobial biofilm cultured in a CDFF.
Species composition of a polymicrobial biofilm in the CDFF (%)

| | Before | At end of | After |
|---|---|---|---|
| | Treatment¹ | treatment² | treatment³ |
| S. mutans | 42 | 51 | 9 |
| S. sanguis | 33 | 21 | 69 |
| A. naeslundii | 7 | 24 | 5.7 |
| L. casei | 2 | 2 | 0.3 |
| F. nucleatum | 16 | 2 | 16 |
| & V. dispar | | | |
| Total | 100 | 100 | 100 |

¹Average % of composition on day 6, 11 & 13. See FIG. 2 for more details.
²% composition on day 25 (KCGPZ(f106-169) treatment from day 18 to 25)
³Average % of composition on day 32 & 33

The clinical efficacy of antimicrobial agents in the oral cavity is largely dependent upon their ability to penetrate or disrupt polymicrobial biofilms accreted to the tooth surface (dental plaque) and kill bacteria that make up these biofilms. The clinical efficacy of antimicrobial agents when used as components of antiseptic mouth rinses is poorly predicted by their effects against planktonic bacteria cells especially if there are long contact times . As a result of this reproducible biofilm models have been developed to test antimicrobial agents. For instance, (Guggenheim et al., 2001) developed a static in vitro polymicrobial biofilm assay that had four oral species representative of supragingival plaque, A. naeslundii, V. dispar, F. nucleatum and Streptococcus sobrinus. These were grown on hydroxyapatite discs in a 24-well plate with processed saliva. To better mimic in vivo conditions, some biofilm models have been designed with a constant supply of fresh, modified or artificial saliva medium. The CDFF system has been used extensively to examine biofilm growth by oral bacteria and to test antimicrobial agents.

Chlorhexidine in 0.1 or 0.2% is considered as a gold standard antibacterial agent in oral care and has been shown to be efficacious against planktonic oral bacterial cells but has produced variable results against oral biofilms. Previous experiments have showed a ˜2 log₁₀ reduction in viable S. sanguis cells grown as a biofilm in a CDFF after a 5 minute exposure to 0.2% w/v chlorhexidine digluconate.. However previous groups have used a CDFF and a complex growth medium containing mucin to culture a six species oral biofilm similar to ours that was dominated by streptococci. These biofilms were treated with 0.2% w/v chlorhexidine digluconate for varying time periods and determined that there was no significant difference after 1 and 5 minutes of exposure. Clinical studies using Chlorhexidine as mouthwash had showed that no difference in results achieved by 0.1% and 0.2% Chlorhexidine digluconate and the 0.12% Chlorhexidine digluconate mouthwash reduced plaque accumulation by 28% and gingival inflammation by 25% over a 12 week period.

The divalent metal ion zinc reduces the growth and metabolism of oral bacteria by interacting with sulfhydryl groups on bacterial enzymes, inhibiting their. activity. The phosphoenolpyruvate:sugar phosphotransferase system and the proton-translocating ATPase are particularly sensitive to zinc inhibition and this inhibition reduces sugar transport and acid tolerance. Zinc is largely bacteriostatic, although very high concentrations can have a bactericidal effect. Its applications are limited by the need to use high concentrations and for oral applications its unpleasant taste and metallic after-taste which are difficult to mask.

Here it has been shown that KCG and KCGPZ(f106-169) are superior to Zinc ions and chlorhexidine in inhibiting S. mutans biofims.

In addition to the traditional cultural analyses to quantitate the effect of antimicrobial agents on bacterial biofilms, CSLM imaging with COMSTAT analysis has been used to visualise and quantitate in vitro biofilms. As shown herein, multi-track flow cell systems were used to reproducibly culture and image S. mutans biofilms and compared the effects of KCG, KCGP(f106-169), KCGPZ(f106-169), chlorhexidine digluconate and zinc chloride on these biofilms. These results show that the biofilm disruptive effects observed could be directly attributed to the glycosylated forms of κ-casein(106-169) as the 2.4 mg/mL KCG treatment produced the same inhibitory effect on the biofilms as 10 mg/mL KCGPZ(f106-169) that contained 2.4 mg/mL KCG (Table 1). The higher concentration of KCG at 10 mg/mL produced a significantly higher reduction in biofilm thickness and biovolume as well as increasing the roughness coefficient (Table 1). Using this method, it was also demonstrated that the distinctive effect of KCGPZ(f106-169) on S. mutans biofilms showing that KCGPZ(f106-169) wase physically disrupting the biofilm (FIG. 1) as KCGPZ(f106-169) reduced S. mutans biofilm biomass and thickness, thus causing a thinner and rougher biofilm with an increased of surface area.

Significant reduction in biofilm biomass and thickness were also detected with treatment with 20 mM ZnCl₂ and 0.1% Chlorhexidine digluconate (Table 1). However, the overall results show that the reduction of the mature 16 h S. mutans biofilm by KCGPZ(f106-169) is distinguished from 0.1% Chlorhexidine digluconate and especially 20 mM ZnCl₂.

In terms of biofilm structures, only S. mutans biofilms treated with the higher concentration of KCG showed a significant difference in surface to biovolume ratio as compared with the control, the data shown herein indicates that KCG and KCGPZ(f106-169) have a disruptive effect on the biofilm. Whereas, the nonsignificant differences in surface to biovolume ratio in the biofilms treated with 0.1% chlorhexidine and 20 mM ZnCl₂ indicate that they do not have a disruptive effect on the biofilms.

The results disclosed in Table 2 suggest that KCGPZ(f106-169) reduces biofilm biomass with disruptive means causing the biofilm to break up into smaller pieces after treatment and subsequent medium flow. The KCGPZ(f106-169) treatment of 6 h biofilm has shown its capability to retard further S. mutans biofilm development (Table 2) rather than a direct antibacterial action.

Considering the efficacy against S. mutans biofilms, KCGPZ(f106-169) was then tested on a supragingival plaque-like multispecies CDFF biofilm cultures and the result indicated that KCGPZ(f106-169) treatment had caused a shift in species composition of the biofilm favoring the less acidogenic species that was still evident seven days after treatment had ceased (Table 3).

Most exopolysaccharides within a biofilm matrix demonstrate a general characteristic of being relatively soluble, forming a highly viscous aqueous solution. Some ions may compete to bind or interact with exposed carboxylic groups of these exopolysaccharides thereby changing the biofilm characteristics; Specifically, without being bound by any theory or mode of action, the zinc ions may enhance the binding of the glycopeptides having N-acetyl neuraminic acid to S. mutans biofilm by their positive charge.

As shown herein, rapidly developing (or immature) monospecific biofilms of S. mutans are highly susceptible to treatment with chlorhexidine digluconate but that more mature biofilms have a more limited response. This may help to explain the varying reports in the literature regarding the effect of chlorhexidine digluconate on oral biofilms as different model systems examine the effects of antimicrobial agents at different points in biofilm development. As also shown herein, KCG and KCGPZ(f106-169) disrupts mature monospecific and polymicrobial biofilm structure and is superior to chlorhexidine in this respect.

Glycosylated κ-casein(106-169) peptides disrupt mature monospecific S. mutans biofilm structures. At relatively low concentrations they have a synergistic effect with zinc ions and together they disrupt polymicrobial oral bacterial biofilms and suppress the recovery of S. mutans.

### EXAMPLE 4

Comparison of antibiofilm efficacy of KCGP(f106-169) with KCP(f106-169).. KCGP(f106-169) [GP] and the non-glycosylated κ-casein (106-169) [KCP(f106-169)] were prepared by chymosin hydrolysis of caseinate and reversed-phase HPLC as described in Example 1 and previously by Malkoski et al (2001) and Dashper et al (2005). The purity of the preparations was confirmed using matrix assisted laser desorption ionisation time of flight mass spectrometry (Malkoski et al, 2001).

Biofilm Assay. E. faecalis strain ATCC 15036 was stored at −20° C. Biofilm antibacterial assays were conducted in sterile 96-well microtiter plates (Becton Dickinson, Franklin Lakes, N.J., USA). Sterile brain heart infusion broth (BHI: 37g/L; Oxoid Ltd., Cambridge, UK) was used as the growth medium for all experiments. Bacteria were enumerated on BHI agar plates as colony forming units (CFU).

The biofilm assay was carried out as described by Malkoski et al (2001) with minor modifications. A 200 μL volume of BHI was added to each well of a 96-well microplate, with 40 μL of experimental solution (ZnCl₂, GP, KCP(f106-169) or NaOCl) and 10 μL of 1×10⁵ E. faecalis cells. Microplates were incubated for 24 h at 37° C. and OD₆₃₀ measured every 15 min using the Microplate, Photometer. At the completion of the 24 h incubation period and after removal of unattached bacterial cells a 1% crystal violet solution was used to stain the biofilm (Wen and Bume, 2002; Loo et al, 2000) which was quantified by colorimetry using a Victor 3™ 1420 Multilabel Counter (Perkin Elmer, Inc., Waltham, Mass., USA) at OD₆₂₀. All experiments were performed in triplicate. Colorimetric readings for each experimental group obtained were statistically analysed using a one-way ANOVA with p<0.05.

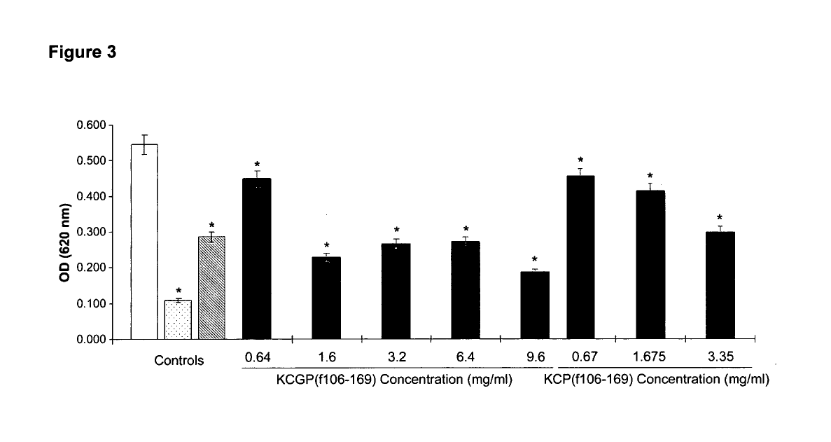

KCGP(f106-169) and KCP(f106-169) at all of the tested concentrations significantly inhibited E. faecalis biofilm formation in comparison to the control culture (p<0.05) (FIG. 3). A dose-dependent increase in the efficacy of biofilm inhibition was noted for KCGP(f106-169) and KCP(f106-169) (FIG. 3). KCGP(f106-169) was significantly (p<0.05) better than KCP(f106-169) at inhibiting biofilm formation at 1.6 mg/mL concentration. Furthermore GP at 1.6 mg/mL to 9.6 mg/mL was significantly better than 10 mg/mL NaOCl at inhibiting E. faecalis biofilm formation.

In this example we show that the casein glycopeptide, KCGP(f106-169), is superior in inhibiting bacterial biofilm when compared with the non-glycosylated casein peptide KCP(f106-169) and the gold standard for treating E. faecalis biofilm, NaOCl.

Compositions and Formulations

To help illustrate compositions embodying aspects of the invention directed to treatment or prevention, the following sample formulations are provided. As used in the following formulations, the phrase “composition or peptide of the invention” includes embodiments of the invention discussed above including peptides with or without a cation.

The following is an example of a toothpaste formulation.

| Ingredient | % w/w |
|---|---|
| Dicalcium phosphate dihydrate | 50.0 |
| Glycerol | 20.0 |
| Sodium carboxymethyl cellulose | 1.0 |
| Sodium lauryl sulphate | 1.5 |
| Sodium lauroyl sarconisate | 0.5 |
| Flavour | 1.0 |
| Sodium saccharin | 0.1 |
| Chlorhexidine gluconate | 0.01 |
| Dextranase | 0.01 |
| Composition or peptide of the invention | 0.2 |
| Water | balance |

The following is an example of a further toothpaste formulation.

| Ingredient | % w/w |
|---|---|
| Dicalcium phosphate dihydrate | 50.0 |
| Sorbitol | 10.0 |
| Glycerol | 10.0 |
| Sodium carboxymethyl cellulose | 1.0 |
| Lauroyl diethanolamide | 1.0 |
| Sucrose monolaurate | 2.0 |
| Flavour | 1.0 |
| Sodium saccharin | 0.1 |
| Sodium monofluorophosphate | 0.3 |
| Chlorhexidine gluconate | 0.01 |
| Dextranase | 0.01 |
| Composition or peptide of the invention | 0.1 |
| Water | balance |

The following is an example of a further toothpaste formulation.

| Ingredient | % w/w |
|---|---|
| Sorbitol | 22.0 |
| Irish moss | 1.0 |
| Sodium Hydroxide (50%) | 1.0 |
| Gantrez | 19.0 |
| Water (deionised) | 2.69 |
| Sodium Monofluorophosphate | 0.76 |
| Sodium saccharine | 0.3 |
| Pyrophosphate | 2.0 |
| Hydrated alumina | 48.0 |
| Flavour oil | 0.95 |
| Composition or peptide of the invention | 0.3 |
| sodium lauryl sulphate | 2.00 |

The following is an example of a liquid toothpaste formulation.

| Ingredient | % w/w |
|---|---|
| Sodium polyacrylate | 50.0 |
| Sorbitol | 10.0 |
| Glycerol | 20.0 |
| Flavour | 1.0 |
| Sodium saccharin | 0.1 |
| Sodium monofluorophosphate | 0.3 |
| Chlorhexidine gluconate | 0.01 |
| Ethanol | 3.0 |
| Composition or peptide of the invention | 0.2 |
| Linolic acid | 0.05 |
| Water | balance |

The following is an example of a mouthwash formulation.

| Ingredient | % w/w |
|---|---|
| Ethanol | 10.0 |
| Flavour | 1.0 |
| Sodium saccharin | 0.1 |
| Sodium monofluorophosphate | 0.3 |
| Chlorhexidine gluconate | 0.01 |
| Lauroyl diethanolamide | 0.3 |
| Composition or peptide of the invention | 0.2 |
| Water | balance |

The following is an example of a further mouthwash formulation useful as an oral lubricant, saliva substitute or as artificial saliva.

| Ingredient | % w/w |
|---|---|
| Gantrez ® S-97 | 2.5 |
| Glycerine | 10.0 |
| Flavour oil | 0.4 |
| Sodium monofluorophosphate | 0.05 |
| Chlorhexidine gluconate | 0.01 |
| Lauroyl diethanolamide | 0.2 |
| Composition or peptide of the invention | 0.3 |
| Water | balance |

The following is an example of a lozenge formulation.

| Ingredient | % w/w |
|---|---|
| Sugar | 75-80 |
| Corn syrup | 1-20 |
| Flavour oil | 1-2 |
| NaF | 0.01-0.05 |
| Composition or peptide of the invention | 0.3 |
| Mg stearate | 1-5 |
| Water | balance |

The following is an example of a gingival massage cream formulation.

| Ingredient | % w/w |
|---|---|
| White petrolatum | 8.0 |
| Propylene glycol | 4.0 |
| Stearyl alcohol | 8.0 |
| Polyethylene Glycol 4000 | 25.0 |
| Polyethylene Glycol 400 | 37.0 |
| Sucrose monostearate | 0.5 |
| Chlorhexidine gluconate | 0.1 |
| Composition or peptide of the invention | 0.3 |
| Water | balance |

The following is an example of a periodontal gel formulation.

| Ingredient | % w/w |
|---|---|
| Pluronic F127 (from BASF) | 20.0 |
| Stearyl alcohol | 8.0 |
| Composition or peptide of the invention | 3.0 |
| Colloidal silicon dioxide (such as Aerosil ® 200 ™) | 1.0 |
| Chlorhexidine gluconate | 0.1 |
| Water | balance |

The following is an example of a chewing gum formulation.

| Ingredient | % w/w |
|---|---|
| Gum base | 30.0 |
| Calcium carbonate | 2.0 |
| Crystalline sorbitol | 53.0 |
| Glycerine | 0.5 |
| Flavour oil | 0.1 |
| Composition or peptide of the invention | 0.3 |
| Water | balance |

It will be understood that the invention disclosed and defined in this specification extends to all alternative combinations of two or more of the individual features mentioned or evident from the text or drawings. All of these different combinations constitute various alternative aspects of the invention.

References

Dashper S G, O'Brien-Simpson N M, Cross K J, Paolini R A, Hoffmann B, Catmull D V, et al. Divalent metal cations increase the activity of the antimicrobial Peptide Kappacin. Antimicrob Agents Chemother 2005; 49:2322-2328.

Guggenheim B, Giertsen E, Schupbach P, Shapiro S (2001). Validation of an in vitro biofilm model of supragingival plaque. J Dent Res 80(1):363-70.

Loo C Y, Corliss D A, Ganeshkumar N. Streptococcus gordonii biofilm formation: identification of genes that code for biofilm phenotypes. J Bacteriol 2000; 182:1374-1382.

Heydorn A, Nielsen A T, Hentzer M, Sternberg C, Givskov M, Ersboll B K, Molin S (2000). Quantification of biofilm structures by the novel computer program COMSTAT. Microbiology 146 2395-407.

Malkoski M, Dashper S G, O'Brien-Simpson N M, Talbo G H, Macris M, Cross K J, et al. Kappacin, a novel antibacterial peptide from bovine milk. Antimicrob Agents Chemother 2001; 45:2309-2315.

McBain A J, Bartolo R G, Catrenich C E, Charbonneau D, Ledder R G, Gilbert P (2003a). Growth and molecular characterization of dental plaque microcosms. J Appl Microbiol 94(4):655-64.

McBain A J, Bartolo R G, Catrenich C E, Charbonneau D, Ledder R G, Gilbert P (2003b). Effects of a chlorhexidine gluconate-containing mouthwash on the vitality and antimicrobial susceptibility of in vitro oral bacterial ecosystems. Appl Environ Microbiol 69(8):4770-6.

Plowman J E, Creamer L K, Liddell M J, Cross J J (1997) Solution conformation of a peptide corresponding to bovine kappa-casein B residues 130-153 by circular dichroism spectroscopy and 1H-nuclear magnetic resonance spectroscopy. J Dairy Sci 64:377-397.

Pratten J, Barnett P, Wilson M (1998). Composition and susceptibility to chlorhexidine of multispecies biofilms of oral bacteria. Appl Environ Microbiol 64(9):3515-9.

Shu M, Browngardt C M, Chen Y Y, Burne R A (2003). Role of urease enzymes in stability of a 10-species oral biofilm consortium cultivated in a constant-depth film fermenter. Infect Immun 71(12):7188-92.

Wen Z T, Burne R A. Functional genomics approach to identifying genes required for biofilm development by Streptococcus mutans. Appl Environ Microbiol 2002; 68:1196-1203.

Wilson M (1996). Susceptibility of oral bacterial biofilms to antimicrobial agents. J Med Microbiol 44(2):79-87.

Wimpenny J, Peters A, Scourfield M (1989). Modeling spatial gradients. In: Structure and function of biofilms. C W. and W P. editors. Chichester, England: John Wiley and Sons, pp. 111-127.

1. A therapeutic composition for treating biofilms consisting essentially of:
a peptide comprising at least one amino acid that is glycosylated, wherein the peptide has an amino acid sequence of, or is functionally similar to, a casein or a fragment of a casein, wherein the fragment is greater than 10 amino acids in length and has at least 70% identity to a portion of a naturally occurring casein;
a divalent cation; and
a pharmaceutically acceptable carrier.

2. The composition according to claim 1, wherein at least one amino acid residue of the peptide is phosphorylated.

3. The composition according to claim 1, wherein the casein is κ-casein.

4. (canceled)

5. The composition according to claim 1, wherein the cation is selected from the group consisting of: Zn²⁺, Ca²⁺, Cu²⁺, Ni²⁺, Co²⁺, Fe²⁺, Sn²⁺ and Mn²⁺.

6. The composition according to claim 5, wherein the cation is Zn²⁺.

7. The composition according to claim 1, wherein the peptide comprises an amino acid sequence according to any one of SEQ ID NO: 1 to 10.

8. (canceled)

9. A saliva substitute consisting essentially of
a therapeutically effective amount of a peptide, each consisting essentially of wherein the peptide comprises an amino acid sequence of, or is functionally similar to, a casein or a fragment of a casein, and wherein the peptide comprises at least one amino acid that is glycosylated;
a divalent cation; and
a pharmaceutically acceptable carrier.

10. (canceled)

11. A method of treating a subject in need comprising administering to the subject a composition according to claim 1.

12. The method according to claim 11, wherein the subject has dental plaque, gingivitis, periodontitis, dental caries, oral mucositis, dry mouth or xerostomia.

13. The method of claim 11, wherein the subject in need requires inhibition, reduction or prevention of a biofilm.

14. The method of claim 11, wherein the subject in need requires biofilm disruption.

15. The composition according to claim 3, wherein the cation is selected from the group consisting of: Zn²⁺, Ca²⁺, Cu²⁺, Ni²⁺, Co²⁺, Fe²⁺, Sn²⁺ and Mn²⁺.

16. The composition according to claim 15, wherein the cation is Zn²⁺.

17. The composition according to claim 16, wherein the peptide comprises an amino acid sequence according to any one of SEQ ID NO: 1 to 10.

18. The saliva substitute according to claim 9, wherein the peptide comprises an amino acid sequence according to any one of SEQ ID NO: 1 to 10.

19. The saliva substitute according to claim 9, wherein the cation is selected from the group consisting of: Zn²⁺, Ca²⁺, Cu²⁺, Ni²⁺, Co²⁺, Fe²⁺, Sn²⁺ and Mn²⁺.

20. The method of claim 11, wherein the composition is formulated as a saliva substitute.


SEQUENCE LISTING (12 sequences)

SEQ ID NO: 1 - protein, 21 aa, Bos taurus
  misc_feature (12)..(12): Xaa is phosphorylated serine
AVESTVATLEAXPEVIESPPE

SEQ ID NO: 2 - protein, 21 aa, Bos taurus
  MISC_FEATURE (12)..(12): Xaa is phosphorylated serine
AVESTVATLEDXPEVIESPPE

SEQ ID NO: 3 - protein, 64 aa, Bos taurus
  MISC_FEATURE (44)..(44): Xaa is phosphorylated serine
MAIPPKKNQDKTEIPTINTIASGEPTSTPTIEAVESTVATLEAXPEVIESPPEINTVQVTSTAV

SEQ ID NO: 4 - protein, 64 aa, Bos taurus
  MISC_FEATURE (22)..(22): Xaa is phosphorylated serine
MAIPPKKNQDKTEIPTINTIAXGEPTSTPTIEAVESTVATLEAXPEVIESPPEINTVQVTSTAV

SEQ ID NO: 5 - protein, 64 aa, Bos taurus
  MISC_FEATURE (44)..(44): Xaa is phosphorylated serine
MAIPPKKNQDKTEIPTINTIASGEPTSTPTTEAVESTVATLEDXPEVIESPPEINTVQVTSTAV

SEQ ID NO: 6 - protein, 64 aa, Bos taurus
  MISC_FEATURE (22)..(22): Xaa is phosphorylated serine
MAIPPKKNQDKTEIPTINTIAXGEPTSTPTTEAVESTVATLEDXPEVIESPPEINTVQVTSTAV

SEQ ID NO: 7 - protein, 53 aa, Bos taurus
  MISC_FEATURE (33)..(33): Xaa is phosphorylated serine
TEIPTINTIASGEPTSTPTIEAVESTVATLEAXPEVIESPPEINTVQVTSTAV

SEQ ID NO: 8 - protein, 53 aa, Bos taurus
  MISC_FEATURE (11)..(11): Xaa is phosphorylated serine
TEIPTINTIAXGEPTSTPTIEAVESTVATLEAXPEVIESPPEINTVQVTSTAV

SEQ ID NO: 9 - protein, 53 aa, Bos taurus
  MISC_FEATURE (33)..(33): Xaa is phosphorylated serine
TEIPTINTIASGEPTSTPTTEAVESTVATLEDXPEVIESPPEINTVQVTSTAV

SEQ ID NO: 10 - protein, 53 aa, Bos taurus
  MISC_FEATURE (11)..(11): Xaa is phosphorylated serine
TEIPTINTIAXGEPTSTPTTEAVESTVATLEDXPEVIESPPEINTVQVTSTAV

SEQ ID NO: 11 - protein, 169 aa, Bos taurus
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 12 - protein, 169 aa, Bos taurus
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV